SEQUENCE LISTING (83 sequences)

SEQ ID NO: 1 - protein, 91 aa, Homo sapiens
VPRDLEVVAATPTSLLISWDAPHHGVAYYRITYGETGGNSPVQEFTVPGSKSTATISGLKPGVDYTINVYAVLAYPRGYPLSKPISINYRT

SEQ ID NO: 2 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 228 aa, Homo sapiens
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 457 aa, Artificial Sequence
  Description of Artificial Sequence Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTLVTVSSASTKGPEVQLVESGGGLVKPGVSLRLSCAASGFTFSRYSMNWVRQAPGKGLEWVSSISSSGTYIKYADSVKGRFTISRDNAKNSLNLQMNSLRAEDTAVYYCARDRDRYPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 7 - protein, 327 aa, Artificial Sequence
  Description of Artificial Sequence Synthetic polypeptide
DIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKRTVAAPSSYELTQPPSVSVSPGQTARITCSGDALPKKYAYWYQQKSGQAPVLVIYEATKRPSGIPERFSGSSSGTMATLTLSGAQVEDEADYYCYSTDSTNYHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 455 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSASTKGPQVQLQESGPGLVKPSETLSLTCTVSGGSIISHYWSWIRQPPGKGLEWIGYIYYSGSTNYNPSLKSRVTISVDTSKNQFSLKLTSVTAADTAVYYCARDGWSAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 9 - protein, 326 aa, Artificial Sequence
  Description of Artificial Sequence Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPDIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 456 aa, Artificial Sequence
  Description of Artificial Sequence Synthetic polypeptide
EVQLVESGGGLVKPGVSLRLSCAASGFTFSRYGMNWVRQAPGKGLEWVSSISSSGTYIKYADSVKGRFTISRDNAKNSLNLQMNSLRAEDTAVYYCARDRDRYPLDYWGQGTLVTVSSASTKGPQVQLQESGPGLVKPSETLSLTCTVSGGSIISHYWSWIRQPPGKGLEWIGYIYYSGSTNYNPSLKSRVTISVDTSKNQFSLKLTSVTAADTAVYYCARDGWSAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAIGLYPYYYRITYGETGGNSPVQEFTVPGSKSTATISGLKPGVDYTITVYAVSGIFGWNNSKPISINYRTGSSDYKDDDDKGSSHHHHHH

SEQ ID NO: 11 - protein, 328 aa, Artificial Sequence
  Description of Artificial Sequence Synthetic polypeptide
SYELTQPPSVSVSPGQTARITCSGDALPKKYAYWYQQKSGQAPVLVIYEATKRPSGIPERFSGSSSGTMATLTLSGAQVEDEADYYCYSTDSTNYHWVFGGGTKLTVLGTVAAPDIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 453 aa, Artificial Sequence
  Description of Artificial Sequence Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTLVTVSSGGGGSGGGGSSSYELTQPPSVSVSPGQTARITCSGDALPKKYAYWYQQKSGQAPVLVIYEATKRPSGIPERFSGSSSGTMATLTLSGAQVEDEADYYCYSTDSTNYHWVFGGGTKLTVLGASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 13 - protein, 341 aa, Artificial Sequence
  Description of Artificial Sequence Synthetic polypeptide
EVQLVESGGGLVKPGVSLRLSCAASGFTFSRYGMNWVRQAPGKGLEWVSSISSSGTYIKYADSVKGRFTISRDNAKNSLNLQMNSLRAEDTAVYYCARDRDRYPLDYWGQGTLVTVSSGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 452 aa, Artificial Sequence
  Description of Artificial Sequence Synthetic polypeptide
SYELTQPPSVSVSPGQTARITCSGDALPKKYAYWYQQKSGQAPVLVIYEATKRPSGIPERFSGSSSGTMATLTLSGAQVEDEADYYCYSTDSTNYHWVFGGGTKLTVLGGGGGSGGGGSQVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 15 - protein, 341 aa, Artificial Sequence
  Description of Artificial Sequence Synthetic polypeptide
DIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKGGGGSGGGGSEVQLVESGGGLVKPGVSLRLSCAASGFTFSRYSMNWVRQAPGKGLEWVSSISSSGTYIKYADSVKGRFTISRDNAKNSLNLQMNSLRAEDTAVYYCARDRDRYPLDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 581 aa, Artificial Sequence
  Description of Artificial Sequence Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTLVTVSSGGGGSGGGGSSSYELTQPPSVSVSPGQTARITCSGDALPKKYAYWYQQKSGQAPVLVIYEATKRPSGIPERFSGSSSGTMATLTLSGAQVEDEADYYCYSTDSTNYHWVFGGGTKLTVLGASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVAAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKQLPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGKAAAHHHHHH

SEQ ID NO: 17 - protein, 576 aa, Artificial Sequence
  Description of Artificial Sequence Synthetic polypeptide
EVQLVESGGGLVKPGVSLRLSCAASGFTFSRYGMNWVRQAPGKGLEWVSSISSSGTYIKYADSVKGRFTISRDNAKNSLNLQMNSLRAEDTAVYYCARDRDRYPLDYWGQGTLVTVSSGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAAAEPKSSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 18 - protein, 470 aa, Artificial Sequence
  Description of Artificial Sequence Synthetic polypeptide
EVQLVESGGGLVKPGVSLRLSCAASGFTFSRYGMNWVRQAPGKGLEWVSSISSSGTYIKYADSVKGRFTISRDNAKNSLNLQMNSLRAEDTAVYYCARDRDRYPLDYWGQGTLVTVSSGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKRAAAEPKSSDKTHTCPPCPAPELLGGPSVFLFPPKPKDTLMISRTPEVTCVVVNVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 19 - protein, 483 aa, Artificial Sequence
  Description of Artificial Sequence Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTLVTVSSGGGGSGGGGSSSYELTQPPSVSVSPGQTARITCSGDALPKKYAYWYQQKSGQAPVLVIYEATKRPSGIPERFSGSSSGTMATLTLSGAQVEDEADYYCYSTDSTNYHWVFGGGTKLTVLGAAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKQLPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGKAAAHHHHHH

SEQ ID NO: 20 - protein, 580 aa, Artificial Sequence
  Description of Artificial Sequence Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 21 - protein, 590 aa, Artificial Sequence [sequence omitted]

SEQ ID NO: 22 - protein, 583 aa, Artificial Sequence
  Description of Artificial Sequence Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSQSVLTQPPSVSEAPRQRVTISCSGSSSNIGNNAVNWYQQLPGKAPKLLIYYDDMLSSGVSDRFSGSKSGTSASLAISGLQSEDEADYYCAAWDDSLNGVVFGGGTKLTVLASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 23 - protein, 587 aa, Artificial Sequence
  Description of Artificial Sequence Synthetic polypeptide
QVQLQESGPGLVKPSQTLSLTCTVSGGSISSGAYYWTWIRQHPGKGLEWIGYIYYSGSTYYNPSLKSRVSISIDTSKNQFSLKLSSVTAADTAVYYCARGSSSWFDYWGQGTLVTVSSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGKAAAHHHHHH

SEQ ID NO: 24 - protein, 456 aa, Artificial Sequence
  Description of Artificial Sequence Synthetic polypeptide
QVQLVESGGGVVQPGRSLRLSCAASGFTFSSYGMHWVRQAPGKGLEWVAVISYPGNTKYYADSVKGRFTISRDNSKNTLYLQMNSLRAEDTAVYFCARDQKEWRLIFDYWGQGTLVTVSSASTKGPQVQLQESGPGLVKPSETLSLTCTVSGGSIISHYWSWIRQPPGKGLEWIGYIYYSGSTNYNPSLKSRVTISVDTSKNQFSLKLTSVTAADTAVYYCARDGWSAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 25 - protein, 327 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGAAFTLTISSLQPEDFATYFCQQANSFPLTFAGGTKVDIKRTVAAPDIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 451 aa, Artificial Sequence
  Description of Artificial Sequence Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTLVTVSSGGGGSGGGGSDIQMTQSPSSVSASVGDRVTITCRASQGISSWLAWYQQKPGKAPKLLIYAASSLQSGVPSRFSGSGSGAAFTLTISSLQPEDFATYFCQQANSFPLTFAGGTKVDIKRASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 27 - protein, 343 aa, Artificial Sequence
  Description of Artificial Sequence Synthetic polypeptide
QVQLVESGGGVVQPGRSLRLSCAASGFTFSSYGMHWVRQAPGKGLEWVAVISYPGNTKYYADSVKGRFTISRDNSKNTLYLQMNSLRAEDTAVYFCARDQKEWRLIFDYWGQGTLVTVSSGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 451 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGAAFTLTISSLQPEDFATYFCQQANSFPLTFAGGTKVDIKRGGGGSGGGGSQVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 29 - protein, 343 aa, Artificial Sequence
  Description of Artificial Sequence Synthetic polypeptide
DIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKGGGGSGGGGSQVQLVESGGGVVQPGRSLRLSCAASGFTFSSYGMHWVRQAPGKGLEWVAVISYPGNTKYYADSVKGRFTISRDNSKNTLYLQMNSLRAEDTAVYFCARDQKEWRLIFDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 463 aa, Artificial Sequence
  Description of Artificial Sequence Synthetic polypeptide
QVQLVESGGGVVQPGRSLRLSCAASGFTFSSYGMHWVRQAPGKGLEWVAVISYPGNTKYYADSVKGRFTISRDNSKNTLYLQMNSLRAEDTAVYFCARDQKEWRLIFDYWGQGTLVTVSSGGGGSGGGGSQVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAVPRDLEVVAATPTSLLISWDAPHHGVAYYRITYGETGGNSPVQEFTVPGSKSTATISGLKPGVDYTINVYAVLAYPRGYPLSKPISINYRTDYKDDDDKGSSHHHHHH

SEQ ID NO: 31 - protein, 331 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGAAFTLTISSLQPEDFATYFCQQANSFPLTFAGGTKVDIKRGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 1371 bp, Artificial Sequence
  Description of Artificial Sequence Synthetic polynucleotide
caggtgcagctgcaggagtcgggcccaggactggtgaagccttcacagaccctgtccctcacctgcactgtctctggtggctccatcaacaataataattactactggacctggatccgccagcacccagggaagggcctggagtggattgggtacatctattacagtgggagcaccttctacaacccgtccctcaagagtcgagttaccatatcagtcgacacgtctaagacccagttctccctgaagttgagctctgtgactgccgcggacacggccgtgtattactgtgcgagagaggatacgatgacgggcctggacgtctggggccaagggaccctggtcaccgtctcctcagcctctacaaagggtcctgaggtgcagctggtggagtctgggggaggcctggtcaagcctggggtgtccctgagactctcctgtgcagcctctggattcaccttcagtagatatagcatgaactgggtccgccaggctccagggaaggggctggagtgggtctcatccattagtagtagtggtacttacataaagtacgcagactcagtgaagggccgattcaccatctccagagacaacgccaagaactcactgaatctgcaaatgaacagcctgagagccgaggacacggctgtgtattattgtgcgagagatcgggaccggtatccccttgactactggggccagggaaccctggtcactgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgccgttccacgtgatttggaagttgttgcagcaactccaactagtctgctgatcagctgggatgcgccgcatcatggtgttgcttattatcgcattacgtacggcgaaaccggcggcaacagcccggtgcaggaattcacggtaccgggcagcaaaagcaccgcgaccatttccggactgaaaccgggcgtggattataccattaacgtgtatgcggtgctggcttacccgcgtggttacccgctgagcaaaccgattagcattaattatcggaccgactacaaagacgatgacgacaagggcagttctcaccatcaccatcaccac

SEQ ID NO: 33 - DNA, 981 bp, Artificial Sequence
  Description of Artificial Sequence Synthetic polynucleotide
gacatccagatgacccagtctccatcctccctgtctgcatctgtaggagacagagtcaccatcacttgccgggctagtcagagcattaacaactatttaaattggtatcagcagaaaccagggaaagcccctacgctcctgatctatgctgcatccagtttgcaaagtggggtcccatcaaggttcagtggcagtagatctgggacagatttcactctcaccatcagcagtctgcaacctgaagattttgcagcttacttctgtcaacagacttacagtaacccgacgttcggccaagggaccaaggtggaagtcaaacgtacggtggctgcaccaagctcctatgagctgacacagccaccctcggtgtcagtgtccccaggacaaacggccaggatcacctgctctggagatgcattgccaaaaaaatatgcttattggtaccagcagaagtcaggccaggcccctgtgctggtcatctatgaggccaccaaacgaccctccgggatccctgagagattctctggctccagctcagggacaatggccaccttgactctcagtggggcccaggtggaggatgaagctgactactactgttactcaacagacagcactaattatcattgggtgttcggcggagggaccaagctgaccgtcctaggccaaccgaaagcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 34 - DNA, 1359 bp, Artificial Sequence
  Description of Artificial Sequence Synthetic polynucleotide
caggtgcagctgcaggagtcgggcccaggactggtgaagccttcacagaccctgtccctcacctgcactgtctctggtggctccatcaacaataataattactactggacctggatccgccagcacccagggaagggcctggagtggattgggtacatctattacagtgggagcaccttctacaacccgtccctcaagagtcgagttaccatatcagtcgacacgtctaagacccagttctccctgaagttgagctctgtgactgccgcggacacggccgtgtattactgtgcgagagaggatacgatgacgggcctggacgtctggggccaagggaccctggtcaccgtctcctcaggaggcggcggttcaggcggaggtggctctagctcctatgagctgacacagccaccctcggtgtcagtgtccccaggacaaacggccaggatcacctgctctggagatgcattgccaaaaaaatatgcttattggtaccagcagaagtcaggccaggcccctgtgctggtcatctatgaggccaccaaacgaccctccgggatccctgagagattctctggctccagctcagggacaatggccaccttgactctcagtggggcccaggtggaggatgaagctgactactactgttactcaacagacagcactaattatcattgggtgttcggcggagggaccaagctgaccgtcctaggcgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgccgttccacgtgatttggaagttgttgcagcaactccaactagtctgctgatcagctgggatgcgccgcatcatggtgttgcttattatcgcattacgtacggcgaaaccggcggcaacagcccggtgcaggaattcacggtaccgggcagcaaaagcaccgcgaccatttccggactgaaaccgggcgtggattataccattaacgtgtatgcggtgctggcttacccgcgtggttacccgctgagcaaaccgattagcattaattatcggaccgactacaaagacgatgacgacaagggcagttctcaccatcaccatcaccac

SEQ ID NO: 35 - DNA, 1023 bp, Artificial Sequence
  Description of Artificial Sequence Synthetic polynucleotide
gaggtgcagctggtggagtctgggggaggcctggtcaagcctggggtgtccctgagactctcctgtgcagcctctggattcaccttcagtagatatggcatgaactgggtccgccaggctccagggaaggggctggagtgggtctcatccattagtagtagtggtacttacataaagtacgcagactcagtgaagggccgattcaccatctccagagacaacgccaagaactcactgaatctgcaaatgaacagcctgagagccgaggacacggctgtgtattattgtgcgagagatcgggaccggtatccccttgactactggggccagggaaccctggtcactgtctcctcaggaggcggcggttcaggcggaggtggctctgacatccagatgacccagtctccatcctccctgtctgcatctgtaggagacagagtcaccatcacttgccgggctagtcagagcattaacaactatttaaattggtatcagcagaaaccagggaaagcccctacgctcctgatctatgctgcatccagtttgcaaagtggggtcccatcaaggttcagtggcagtagatctgggacagatttcactctcaccatcagcagtctgcaacctgaagattttgcagcttacttctgtcaacagacttacagtaacccgacgttcggccaagggaccaaggtggaagtcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 36 - DNA, 1356 bp, Artificial Sequence
  Description of Artificial Sequence Synthetic polynucleotide
tcctatgagctgacacagccaccctcggtgtcagtgtccccaggacaaacggccaggatcacctgctctggagatgcattgccaaaaaaatatgcttattggtaccagcagaagtcaggccaggcccctgtgctggtcatctatgaggccaccaaacgaccctccgggatccctgagagattctctggctccagctcagggacaatggccaccttgactctcagtggggcccaggtggaggatgaagctgactactactgttactcaacagacagcactaattatcattgggtgttcggcggagggaccaagctgaccgtcctaggcggaggcggcggttcaggcggaggtggctctcaggtgcagctgcaggagtcgggcccaggactggtgaagccttcacagaccctgtccctcacctgcactgtctctggtggctccatcaacaataataattactactggacctggatccgccagcacccagggaagggcctggagtggattgggtacatctattacagtgggagcaccttctacaacccgtccctcaagagtcgagttaccatatcagtcgacacgtctaagacccagttctccctgaagttgagctctgtgactgccgcggacacggccgtgtattactgtgcgagagaggatacgatgacgggcctggacgtctggggccaagggaccacggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgccgttccacgtgatttggaagttgttgcagcaactccaactagtctgctgatcagctgggatgcgccgcatcatggtgttgcttattatcgcattacgtacggcgaaaccggcggcaacagcccggtgcaggaattcacggtaccgggcagcaaaagcaccgcgaccatttccggactgaaaccgggcgtggattataccattaacgtgtatgcggtgctggcttacccgcgtggttacccgctgagcaaaccgattagcattaattatcggaccgactacaaagacgatgacgacaagggcagttctcaccatcaccatcaccac

SEQ ID NO: 37 - DNA, 1023 bp, Artificial Sequence
  Description of Artificial Sequence Synthetic polynucleotide
gacatccagatgacccagtctccatcctccctgtctgcatctgtaggagacagagtcaccatcacttgccgggctagtcagagcattaacaactatttaaattggtatcagcagaaaccagggaaagcccctacgctcctgatctatgctgcatccagtttgcaaagtggggtcccatcaaggttcagtggcagtagatctgggacagatttcactctcaccatcagcagtctgcaacctgaagattttgcagcttacttctgtcaacagacttacagtaacccgacgttcggccaagggaccaaggtggaagtcaaaggaggcggcggttcaggcggaggtggctctgaggtgcagctggtggagtctgggggaggcctggtcaagcctggggtgtccctgagactctcctgtgcagcctctggattcaccttcagtagatatagcatgaactgggtccgccaggctccagggaaggggctggagtgggtctcatccattagtagtagtggtacttacataaagtacgcagactcagtgaagggccgattcaccatctccagagacaacgccaagaactcactgaatctgcaaatgaacagcctgagagccgaggacacggctgtgtattattgtgcgagagatcgggaccggtatccccttgactactggggccagggaaccctggtcactgtctcctcacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 38 - DNA, 1749 bp, Artificial Sequence
  Description of Artificial Sequence Synthetic polynucleotide
gaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggaggcggcggttcaggcggaggtggctctcagtctgtgctgactcagccaccctcggtgtctgaagcccccaggcagagggtcaccatctcctgttctggaagcagctccaacatcggaaataatgctgtaaactggtaccagcagctcccaggaaaggctcccaaactcctcatctattatgatgatatgttgtcttcaggggtctcggaccgattttctggctccaagtctggcacctcagcctccctggccatcagtgggctccagtctgaggatgaggctgattattactgtgcagcatgggatgacagcctgaatggtgtggtattcggcggagggaccaagctgaccgtcctagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 39 - DNA, 1761 bp, Artificial Sequence
  Description of Artificial Sequence Synthetic polynucleotide
caggtgcagctgcaggagtcgggcccaggactggtgaagccttcacagaccctgtccctcacctgcactgtctctggtggctccatcagcagtggtgcttactactggacctggatccgccagcacccagggaagggcctggagtggattgggtacatctattacagtgggagcacctactacaacccgtccctcaagagtcgagttagcatatcaatagacacgtctaagaaccagttctccctgaagctgagctctgtgactgccgcggacacggccgtgtattactgtgcgcgaggcagcagcagctggttcgactactggggccagggaaccctggtcaccgtctcctcaggaggcggcggttcaggcggaggtggctctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcagcggccgcagagcccaaatcttctgacaaaactcacacatgccccccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagctgcagcgcatcaccaccaccatcac

SEQ ID NO: 40 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 41 - protein, 177 aa, Macaca fascicularis
QDGNEEMGSITQTPYQVSISGTTVILTCSQHLGSEAQWQHNGKNKGDSGDQLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 42 - protein, 118 aa, Artificial Sequence
  Description of Artificial Sequence Synthetic polypeptide
EVQLVESGGGLVKPGVSLRLSCAASGFTFSRYSMNWVRQAPGKGLEWVSSISSSGTYIKYADSVKGRFTISRDNAKNSLNLQMNSLRAEDTAVYYCARDRDRYPLDYWGQGTLVTVSS

SEQ ID NO: 43 - protein, 109 aa, Artificial Sequence
  Description of Artificial Sequence Synthetic polypeptide
SYELTQPPSVSVSPGQTARITCSGDALPKKYAYWYQQKSGQAPVLVIYEATKRPSGIPERFSGSSSGTMATLTLSGAQVEDEADYYCYSTDSTNYHWVFGGGTKLTVLG

SEQ ID NO: 44 - protein, 125 aa, Artificial Sequence
  Description of Artificial Sequence Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 45 - protein, 109 aa, Artificial Sequence
  Description of Artificial Sequence Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 46 - protein, 119 aa, Artificial Sequence
  Description of Artificial Sequence Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTLVTVSS

SEQ ID NO: 47 - protein, 118 aa, Artificial Sequence
  Description of Artificial Sequence Synthetic polypeptide
EVQLVESGGGLVKPGVSLRLSCAASGFTFSRYGMNWVRQAPGKGLEWVSSISSSGTYIKYADSVKGRFTISRDNAKNSLNLQMNSLRAEDTAVYYCARDRDRYPLDYWGQGTLVTVSS

SEQ ID NO: 48 - protein, 107 aa, Artificial Sequence
  Description of Artificial Sequence Synthetic polypeptide
DIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKR

SEQ ID NO: 49 - protein, 119 aa, Artificial Sequence
  Description of Artificial Sequence Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSS

SEQ ID NO: 50 - protein, 108 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGAAFTLTISSLQPEDFATYFCQQANSFPLTFAGGTKVDIKR

SEQ ID NO: 51 - protein, 120 aa, Artificial Sequence
  Description of Artificial Sequence Synthetic polypeptide
QVQLVESGGGVVQPGRSLRLSCAASGFTFSSYGMHWVRQAPGKGLEWVAVISYPGNTKYYADSVKGRFTISRDNSKNTLYLQMNSLRAEDTAVYFCARDQKEWRLIFDYWGQGTLVTVSS

SEQ ID NO: 52 - protein, 110 aa, Artificial Sequence
  Description of Artificial Sequence Synthetic polypeptide
QSVLTQPPSVSEAPRQRVTISCSGSSSNIGNNAVNWYQQLPGKAPKLLIYYDDMLSSGVSDRFSGSKSGTSASLAISGLQSEDEADYYCAAWDDSLNGVVFGGGTKLTVL

SEQ ID NO: 53 - protein, 118 aa, Artificial Sequence
  Description of Artificial Sequence Synthetic polypeptide
QVQLQESGPGLVKPSQTLSLTCTVSGGSISSGAYYWTWIRQHPGKGLEWIGYIYYSGSTYYNPSLKSRVSISIDTSKNQFSLKLSSVTAADTAVYYCARGSSSWFDYWGQGTLVTVSS

SEQ ID NO: 54 - protein, 12 aa, Artificial Sequence
  Description of Artificial Sequence Synthetic peptide
GGCVFNMFNCGG

SEQ ID NO: 55 - protein, 12 aa, Artificial Sequence
  Description of Artificial Sequence Synthetic peptide
GGCHLPFAVCGG

SEQ ID NO: 56 - protein, 12 aa, Artificial Sequence
  Description of Artificial Sequence Synthetic peptide
GGCGHEYMWCGG

SEQ ID NO: 57 - protein, 12 aa, Artificial Sequence
  Description of Artificial Sequence Synthetic peptide
GGCWPLQDYCGG

SEQ ID NO: 58 - protein, 12 aa, Artificial Sequence
  Description of Artificial Sequence Synthetic peptide
GGCMQMNKWCGG

SEQ ID NO: 59 - protein, 12 aa, Artificial Sequence
  Description of Artificial Sequence Synthetic peptide
GGCDGRTKYCGG

SEQ ID NO: 60 - protein, 12 aa, Artificial Sequence
  Description of Artificial Sequence Synthetic peptide
GGCALYPTNCGG

SEQ ID NO: 61 - protein, 12 aa, Artificial Sequence
  Description of Artificial Sequence Synthetic peptide
GGCGKHWHQCGG

SEQ ID NO: 62 - protein, 12 aa, Artificial Sequence
  Description of Artificial Sequence Synthetic peptide
GGCHSFKHFCGG

SEQ ID NO: 63 - protein, 12 aa, Artificial Sequence
  Description of Artificial Sequence Synthetic peptide
GGCQGMWTWCGG

SEQ ID NO: 64 - protein, 14 aa, Artificial Sequence
  Description of Artificial Sequence Synthetic peptide
GGCAQQWHHEYCGG

SEQ ID NO: 65 - protein, 12 aa, Artificial Sequence
  Description of Artificial Sequence Synthetic peptide
GGCERFHHACGG

SEQ ID NO: 66 - protein, 5 aa, Artificial Sequence
  Description of Artificial Sequence Synthetic peptide
TVAAP

SEQ ID NO: 67 - protein, 6 aa, Artificial Sequence
  Description of Artificial Sequence Synthetic peptide
ASTKGP

SEQ ID NO: 68 - protein, 10 aa, Artificial Sequence
  Description of Artificial Sequence Synthetic peptide
GGGGSGGGGS

SEQ ID NO: 69 - protein, 8 aa, Artificial Sequence
  Description of Artificial Sequence Synthetic peptide
GGGGSAAA

SEQ ID NO: 70 - protein, 98 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 71 - protein, 105 aa, Artificial Sequence
  Description of Artificial Sequence Synthetic polypeptide
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 72 - protein, 107 aa, Artificial Sequence
  Description of Artificial Sequence Synthetic polypeptide
DIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIR

SEQ ID NO: 73 - protein, 107 aa, Artificial Sequence [sequence omitted]

SEQ ID NO: 74 - protein, 15 aa, Artificial Sequence
  Description of Artificial Sequence Synthetic peptide
GGGGSGGGGSGGGGS

SEQ ID NO: 75 - protein, 518 aa, Artificial Sequence
  Description of Artificial Sequence Synthetic polypeptide
EVQLLEQSGAELVRPGALVKLSCKASGFKIKDYFVNWVKQRPEQGLEWIGWIDPENDNSLYGPNFQDKASITADTSSNTGYLQLSGLTSEDTAVYYCALYYGSRGDAMDYWGQGTTVTVSSGGGGSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAADYKDDDDKGSSHHHHHH

SEQ ID NO: 76 - protein, 518 aa, Artificial Sequence
  Description of Artificial Sequence Synthetic polypeptide
QVQLQESGPGLVKPSQTLSLTCTVSGGSISSGAYYWTWIRQHPGKGLEWIGYIYYSGSTYYNPSLKSRVSISIDTSKNQFSLKLSSVTAADTAVYYCARGSSSWFDYWGQGTLVTVSSGGGGSGGGGSGGGGSQSVLTQPPSVSEAPRQRVTISCSGSSSNIGNNAVNWYQQLPGKAPKLLIYYDDMLSSGVSDRFSGSKSGTSASLAISGLQSEDEADYYCAAWDDSLNGVVFGGGTKLTVLSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAADYKDDDDKGSSHHHHHH

SEQ ID NO: 77 - protein, 9 aa, Artificial Sequence
  Description of Artificial Sequence Synthetic peptide
  MOD_RES (2)..(9): Any amino acid
CXXXXXXXX

SEQ ID NO: 78 - protein, 5 aa, Artificial Sequence
  Description of Artificial Sequence Synthetic peptide
LEWIG

SEQ ID NO: 79 - protein, 4 aa, Artificial Sequence
  Description of Artificial Sequence Synthetic peptide
  MOD_RES (3)..(3): Any amino acid
WGXG

SEQ ID NO: 80 - protein, 4 aa, Artificial Sequence
  Description of Artificial Sequence Synthetic peptide
  MOD_RES (3)..(3): Any amino acid
FGXG

SEQ ID NO: 81 - protein, 5 aa, Artificial Sequence
  Description of Artificial Sequence Synthetic peptide
QDGNE

SEQ ID NO: 82 - protein, 125 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 83 - protein, 109 aa, Artificial Sequence; identical to SEQ ID NO: 45
  Description of Artificial Sequence Synthetic polypeptide

(19) United States
(12) Patent Application Publication
(10) Pub. No.: US 2023/0077648 A1
(43) Pub. Date: Mar. 16, 2023

(54) Heterodimeric Bispecific Antibodies

(71) Applicant: AMGEN INC., Thousand Oaks, CA (US)

(72) Inventors: Wei Yan, Thousand Oaks, CA (US); Martin J. Pentony, Seattle, WA (US); Luis G. Borges, Redwood City, CA (US); Mark L. Michaels, Encino, CA (US)

(21) Appl. No.: 17/722,724

(22) Filed: Apr. 18, 2022

Related U.S. Application Data
(63) Continuation of application No. 16/383,115, filed on Apr. 12, 2019, now abandoned.
Application No. 16/383,115 is a continuation of application No. 14/207,368, filed on Mar. 12, 2014, now abandoned.
(60) Provisional application No. 61/791,357, filed on Mar. 15, 2013.

(51) Int. Cl. C07K 16/46; C07K 16/30; C07K 16/28; C07K 16/32

(52) U.S. Cl. CPC C07K 16/468; C07K 16/3069; C07K 16/28; C07K 16/2809; C07K 16/32; A61K 2039/505

(57) ABSTRACT

Provided herein are heterodimeric bispecific antibodies that can mediate cytolysis of a target cell by an immune effector cell, nucleic acids encoding such antibodies, methods of making such antibodies, and methods of using such antibodies. These antibodies comprise two different polypeptide chains, each comprising two immunoglobulin variable regions and, optionally, a half life-extending moiety.

### CROSS REFERENCE TO RELATED APPLICATIONS

This application claims the benefit of U.S. Provisional Application 61/791,357, filed Mar. 15, 2013, the content of which is incorporated herein in its entirety.

### Sequence Listing

The instant application contains a Sequence Listing which has been submitted electronically in ASCII format and is hereby incorporated by reference in its entirety. Said ASCII copy, created on Mar. 5, 2014, is named A-1809-US-NP_SL.txt and is 165,079 bytes in size.

### FIELD

The invention is in the field of antibody engineering.

### BACKGROUND

Bispecific antibodies have a lot of promise as therapeutics in a variety of indications. Bispecific antibodies having a standard IgG format can be challenging to produce because they include four different polypeptide chains. The efficacy of a smaller, more easily-produced bispecific molecule has been clinically demonstrated in non-Hodgkin's lymphoma. See, e.g., Bargou et al. (2008), Science 321(5891): 974-977. Daily administration was used to achieve these results, presumably because of the short in vivo half life of this single chain molecule. Id. Hence, there is a need in the art for bispecific therapeutics with favorable pharmacokinetic properties, as well as therapeutic efficacy and a format that makes them straightforward to produce.

### SUMMARY

The bispecific heterodimeric antibody format described herein produces an antibody that can bind to one molecule of each of two different proteins and contains a half-life extending moiety, for example, an Fc region of an antibody. Thus, the bispecific antibody itself will not directly cause the multimerization of either of the proteins on a cell surface. The antibody also can have favorable pharmacokinetic properties relative to a molecule lacking a half-life extending moiety. In some embodiments, one protein bound by the antibody is expressed on an immune effector cell, such as a T cell or an NK cell, and the other protein is expressed on a target cell, for example, a cancer cell. Multimerization of certain proteins expressed on immune effector cells causes a generalized activation of the immune effector cell, a situation that could potentially cause undesirable, generalized inflammation. The bispecific heterodimeric antibodies described herein have desirable pharmacokinetic properties and can bind to two specific proteins, one of which is expressed on an immune effector cell and the other of which is expressed on a diseased cell, such as a cancer cell. The binding of the bispecific heterodimeric antibody brings the immune effector cell and the target cell together and induces the immune effector cell to eliminate the target cell, likely through a mechanism similar to that observed with some other bispecific antibodies. See, e.g., Hass et al. (2009), Immunobiology 214(6): 441-53.

In one aspect, provided herein is heterodimeric bispecific antibody comprising (a) a first polypeptide chain having the formula V1-L1-V2-L2-CH1, wherein V1 and V2 are immunoglobulin variable regions, L1 and L2 are linkers, L2 can be present or absent, and CH1 is a first immunoglobulin heavy chain constant region; and (b) a second polypeptide chain having the formula V3-L3-V4-L4-CL, wherein V3 and V4 are immunoglobulin variable regions, L3 and L4 are linkers, L4 can be present or absent, and CL is an immunoglobulin light chain constant region; wherein either or both of the first and the second polypeptide chains further comprise(s) a half life-extending moiety downstream from the regions recited in (a) and (b); wherein V1, V2, V3, and V4 have different amino acid sequences; and wherein the heterodimeric bispecific antibody mediates cytolysis of a target cell displaying a target cell protein by an immune effector cell, but does not mediate the cytolysis of a cell that does not display a target cell protein by the immune effector cell and/or the heterodimeric antibody binds to a target cell and an immune effector cell. The half life-extending moiety can be a polypeptide. A half life—extending moiety can be downstream from the regions recited in (a) and/or from the regions recited in (b). The half life-extending moiety can be an Fc polypeptide chain, and the first and second polypeptide chains can each comprise an Fc polypeptide chain downstream from the regions recited in (a) and (b). The target cell can be a cancer cell. The immune effector cell can be a T cell, an NK cell, a macrophage, or a neutrophil, and the heterodimeric bispecific antibody can mediate increased expression of CD25 and CD69 on the T cell in the presence of target cells, but not in the absence of target cells. The Fc polypeptide chains of the first and second polypeptide chains can be human IgG Fc polypeptide chains, such as IgG1, IgG2, IgG3, or IgG4 Fc polypeptide chains or variants thereof comprising not more than 10 deletions, insertions, or substitutions of a single amino acid per 100 amino acids of sequence. In some embodiments, L1 and L3 are no more than 12 amino acids long or 10 amino acids long. In some embodiments, one of V1 and V4 can be an immunoglobulin heavy chain variable (VH) region and the other can be an immunoglobulin light chain variable (VL) region, and V1 and V4 can bind to a target cell or an immune effector cell when they are part of an IgG or and/or an scFv antibody. In such embodiments, one of V2 and V3 can be a VH region and the other can be a VL region, and V2 and V3 can bind to a target cell or an immune effector cell when they are part of an IgG and/or an scFv antibody. V1 and V3 can be VL regions, and V2 and V4 can be VH regions. In other embodiments, V1 and V3 can be VH regions, and V2 and V4 can be VL regions. In futher embodiments, V1 and V2 can be VL regions, and V3 and V4 can be VH regions. In still other embodiments, V1 and V2 can be VH regions, and V3 and V4 can be VL regions.

In another aspect, one of V1 and V3 can be a VH region and the other can be a VL region, and V1 and V3 can bind to a target cell or an immune effector cell when they are part of an IgG and/or an scFv antibody. In such embodiments, one of V2 and V4 can be a VH region and the other can be a VL region, and V2 and V4 can bind to a target cell or an immune effector cell when they are part of an IgG and/or an scFv antibody. In a further aspect, V1 and V2 can be VH regions, and V3 and V4 can be VL regions. Alternatively, V1 and V2 can be VL regions, and V3 and V4 can be VH regions. In another aspect, V1 and V4 can be VH regions, and V2 and V3 can be VL regions. In a further aspect, V1 and V4 can be VL regions, and V2 and V3 can be VH regions.

Any heterodimeric bispecific antibody described herein can bind to an immune effector cell. The effector cell protein can be part of a human TCR-CD3 complex. In such a case, the effector cell protein can be the CD3c chain.

In another aspect, a heterodimeric bispecific antibody can comprise a VH region comprising the amino acid sequence of SEQ ID NO:42 or a variant of SEQ ID NO:42 containing not more than 20 insertions, deletions, or substitutions relative to SEQ ID NO:42 and a VL region comprising the amino acid sequence of SEQ ID NO:43 or a variant of SEQ ID NO:43 containing not more than 20 insertions, deletions, or substitutions of a single amino acid relative to SEQ ID NO:43. Alternatively, a heterodimeric bispecific antibody can comprise a VH region comprising the amino acid sequence of SEQ ID NO:44 or a variant of SEQ ID NO:44 containing not more than 20 insertions, deletions, or substitutions relative to SEQ ID NO:44 and a VL region comprising the amino acid sequence of SEQ ID NO:45 or a variant of SEQ ID NO:45 containing not more than 20 insertions, deletions, or substitutions of a single amino acid relative to SEQ ID NO:45 . In other embodiments, a heterodimeric bispecific antibody can comprise a V1, V2, V3, and V4 that comprise the amino acid sequences of SEQ ID NO:46, SEQ ID NO:43, SEQ ID NO:47, and SEQ ID NO:48, respectively. Alternatively, a heterodimeric bispecific antibody can comprise a V1, V2, V3, and V4 that comprise the amino acid sequences of SEQ ID NO:43, SEQ ID NO:49, SEQ ID NO:48, and SEQ ID NO:42, respectively. In a further alternative, a heterodimeric bispecific antibody can comprise a V1, V2, V3, and V4 that comprise the amino acid sequences of SEQ ID NO:50, SEQ ID NO:49, SEQ ID NO:48, and SEQ ID NO:51, respectively. In still another alternative, a heterodimeric bispecific antibody can comprise a V1, V2, V3, and V4 that comprise the amino acid sequences of SEQ ID NO:4, SEQ ID NO:52, SEQ ID NO:53, and SEQ ID NO:45, respectively. In the constructs mentioned above, the VH and VL regions having the amino acid sequences of SEQ ID NOs:82 and 83, respectively, can replace the VH and VL regions SEQ ID NOs:42 and 43 or SEQ ID NOs:44 and 45. Any heterodimeric bispecific antibody described herein can comprise the amino acid sequences of SEQ ID NO:82 and 83. It is further contemplated that variants of the amino acid sequences mentioned above containing not more than 10 deletions, insertions, or substitutions of a single amino acid per 100 amino acids of sequence are provided herein.

Any heterodimeric bispecific antibody described herein that comprises an Fc polypeptide chain on both the first and second polypeptide chains can comprise at least one charge pair substitution on each Fc polypeptide chain. In some such embodiments, the Fc polypeptide chain portion of the first polypeptide chain can comprise the charge pair substitutions D356K or D356R and D399K or D399R, and the Fc polypeptide chain portion of the second polypeptide can comprise the charge pair substitutions K409D or K409E and K392D or K392E. In other such embodiments, the Fc polypeptide chain portion of the second polypeptide chain can comprise the charge pair substitutions D356K or D356R and D399K or D399R, and the Fc polypeptide chain portion of the first polypeptide comprises the charge pair substitutions K409D or K409E and K392D or K392E.

Any heterodimeric bispecific antibody described herein that comprises an Fc polypeptide chain on both the first and second polypeptide chains can comprise one or more alterations that inhibit Fc gamma receptor (FcγR) binding. Such alterations can include L234A, L235A, and/or any substitution at position 297.

Any heterodimeric bispecific antibody described herein that comprises an Fc polypeptide chain on both the first and second polypeptide chains can comprise one or more Fc alterations that extend half life. Such alterations can include an insertion between residues 384 and 385, according to the EU numbering system, in each of the Fc polypeptide chain portions of the first and second polypeptide chains, wherein the insertion comprises the amino acid sequence of any one of SEQ ID NOs:62-73.

In another aspect, any heterodimeric bispecific antibody described herein that comprises an Fc polypeptide chain on both the first and second polypeptide chains can comprise one or more alterations that enhance ADCC in the Fc polypeptide chain portions of the first and second polypeptide chains.

In addition, provided herein are one or more nucleic acid(s) encoding any polypeptide chain of any of the heterodimeric bispecific antibodies described herein. Exemplary nucleic acid sequences include SEQ ID NOs:32, 33, 34, 35, 36, 37, 38, and 39. Further provided are one or more vector(s) comprising such nucleic acid(s), and host cells containing such nucleic acid(s) or vector(s). In another aspect, described herein are methods of making a heterodimeric bispecific antibody comprising culturing a host cell containing such nucleic acids under conditions so as to express the nucleic acid encoding the heterodimeric bispecific antibody and recovering the antibody from the cell mass or cell culture supernatant.

In a different aspect, described herein is a method of treating a cancer patient comprising administering to the patient a therapeutically effective amount of any heterodimeric bispecific antibody described herein, wherein the target cell protein is a cancer cell antigen. In some embodiments, chemotherapy or radiation can be administered to the patient concurrently with, before, or after administration of the antibody. In another approach, a non-chemotherapeutic anti-neoplastic agent can be administered to the patient concurrently with, before, or after administration of the antibody.

In a further aspect, described herein is method for treating a patient having an infectious disease comprising administering to the patient a therapeutically effective dose of any heterodimeric bispecific antibody described herein, wherein the target cell is an infected cell.

In a further aspect, provided herein is method for treating a patient having an autoimmune or inflammatory condition or a fibrotic condition comprising administering to the patient a therapeutically effective dose of any heterodimeric bispecific antibody described herein.

Provided herein is a use of any heterodimeric bispecific antibody described herein as a medicament.

In a further aspect, described herein is a pharmaceutical composition comprising any heterodimeric bispecific antibody described herein. The pharmaceutical composition can be for the treatment of cancer, an infectious disease, an autoimmune or inflammatory disease, or a fibrotic disease.

### BRIEF DESCRIPTION OF THE FIGURES

FIG. 1: Exemplary subtypes of heterodimeric bispecific antibodies. In these diagrams VH1 and VL1 are a pair of immunoglobulin heavy and light chain variable regions that can bind to a “target cell protein,” and VH2 and VL2 are a pair of immunoglobulin heavy and light chain variable regions that can bind to an “effector cell protein.” Other regions depicted in the diagrams are identified in the figure. The dashed lines surrounding the CL and CH1 regions mean that these regions can be eliminated in some embodiments. In some embodiments, both the CL and the CH1 regions are eliminated. The dashed lines delineating the squares representing the half life-extending moieties also indicate that these can be eliminated in some embodiments. However, in this case, only one or the other, not both, half life-extending moieties can be eliminated.

FIG. 2: Heterodimeric bispecific anti-MSLN/CD3 antibodies induce lysis of MSLN-expressing tumor cell lines in the presence of human T cells. The x axis indicates the antibody concentration (log nM), and the y axis indication the percent specific cell lysis. All methods are described in Example 2, and the particular heterodimeric bispecific antibody constructs used are indicated in the figure.

FIG. 3: Heterodimeric bispecific anti-MSLN/CD3 antibodies induce lysis of MSLN-expressing tumor cell lines in the presence of human T cells. The x axis indicates the antibody concentration (log nM), and the y axis indication the percent specific cell lysis. All methods are described in Example 2, and the particular heterodimeric bispecific antibody constructs used are indicated in the figure.

FIG. 4: Heterodimeric bispecific anti-MSLN/CD3 antibodies induce lysis of MSLN-expressing tumor cell lines in the presence of cynomolgus monkey T cells. The x axis indicates the antibody concentration (log nM), and the y axis indication the percent specific cell lysis. All methods are described in Example 2, and the particular heterodimeric bispecific antibody constructs used are indicated in the figure.

FIG. 5: Bispecific anti-MSLN/CD3 antibodies in various formats induce lysis of MSLN-expressing tumor cell lines in the presence of human T cells. The x axis indicates the antibody concentration (log nM), and the y axis indication the percent specific cell lysis. All methods are described in Example 3, and the particular heterodimeric bispecific antibody constructs used are indicated in the figure.

FIG. 6: A heterodimeric bispecific anti-HER2/CD3 antibody (P136797.3 , solidly filled circles and solid lines) and anti-HER2/CD3 single chain bispecific molecule (P136629.3 , open circles and dashed lines) induces lysis of HER2-expressing tumor cell lines (JIMT-1 and T47D) in the presence of human T cells. The x axis indicates antibody concentration (pM), and the y axis indicates percent specific cell lysis. The cell line used, i.e., JIMT-1, T47D, or SHP77 (which does not express HER2), is indicated in each panel. Methods are disclosed in Example 4.

FIG. 7: Peripheral CD3⁺ T cells show CD25 and CD69 up-regulation in response to anti-HER2/CD3 heterodimeric bispecfic antibody or single chain anti-HER2/CD3 bispecific antibody treatment in the presence of HER2-expressing tumor target cells. Expression of CD25 (left panel) and CD69 (right panel) in CD3⁺ peripheral blood T cells was measured by fluorescence activated cell sorting (FACS) as explained in Example 5. The x axis indicates the concentration of the anti-HER2/CD3 heterodimeric bispecific antibody (P1367973) or the single chain anti-HER2/CD3 bispecific antibody (P136629.3) (pM) in both panels, and the y axis indicates the percent of CD3⁺ cells that were also CD25 positive (left panel) or CD69 positive (right panel). Symbols indicate as follows: open squares connected by dashed lines, single chain anti-HER2/CD3 bispecific antibody with tumor target cells; filled, downward pointed triangles connected by solid lines, anti-HER2/CD3 heterodimeric bispecfic antibody with tumor target cells; open circles connected by dashed lines, single chain anti-HER2/CD3 bispecific antibody without tumor target cells; and filled, upward pointing triangles, anti-HER2/CD3 heterodimeric bispecfic antibody without tumor target cells.

FIG. 8: Heterodimeric anti-FOLR1/CD3 heterodimeric bispecific antibody (solidly filled circles and solid lines) or single chain anti-FOLR1/CD3 molecule (open circles and dashed lines) induces lysis of FOLR1-expressing tumor cell lines. The x axis indicates the concentration of the heterodimeric anti-FOLR1/CD3 bispecific antibody or anti-FOLR1/CD3 single chain molecule (pM), and the y axis indicates the percent of tumor target cells lysed. Methods are described in Example 6. As indicated, data from the Cal-51, T47D, and BT474 cell lines are in the top, middle, and bottom panels, respectively.

FIGS. 9A-9B: An anti-FOLR1/CD3 heterodimeric bispecific antibody or single chain anti-FOLR1/CD3 molecule stimulates release of cytokines from T cells in the presence of a FOLR1-expressing tumor cell line (T47D). The methods used are described in Example 6. In each panel, the x axis indicates the concentration of the anti-FOLR1/CD3 heterodimeric bispecific antibody or single chain molecule (pM) used in the TDCC assay. The y axis indicates the concentration of the cytokine detected in the supernatant (pg/mL). Open circles connected by a dashed line indicate data from samples containing the anti-FOLR1/CD3 heterodimeric bispecific antibody, whereas solidly filled circles connected by solid lines indicate data from samples containing the anti-FOLR1/CD3 single chain molecule. The cytokines assayed are indicated in each panel. As indicated, panels on the left show data from samples containing T47D cells, and panels on the right show data from samples containing BT474 cells. As indicated, FIG. 9A shows data on interferon gamma (IFNγ, top), tumor necrosis factor alpha (TNFα, middle), and interleukin-10 (IL-10, bottom), and FIG. 9B shows data on interleukin-2 (IL-2, top) and interleukin-13 (IL-13, bottom).

FIGS. 10A-10B: An anti-HER2/CD3 heterodimeric bispecific antibody or anti-HER2/CD3 single chain molecule stimulates the release of cytokines from T cells in the presence of a HER2-expressing tumor cell line (JIMT-1). The methods used are described in Example 7. In each panel, the x axis indicates the concentration of the anti-HER2/CD3 heterodimeric bispecific antibody or single chain molecule (pM) used in the TDCC assay. The y axis indicates the concentration of the cytokine detected in the supernatant (pg/mL). Open circles connected by a dashed line indicate data from samples containing the anti-HER2/CD3 heterodimeric bispecific antibody, whereas solidly filled circles connected by solid lines indicate data from samples containing the anti-HER2/CD3 single chain molecule. The cytokines assayed are indicated in each panel. As indicated, panels on the left show data from samples containing JIMT-1 cells, and panels on the right show data from samples containing SHP77 cells. As indicated, FIG. 10A shows data on IFNy (top), TNFα (middle), and IL-10 (bottom), and FIG. 10B shows data on IL-2 (top) and IL-13 (bottom).

FIG. 11: In vivo inhibition of tumor growth by an anti-MSLN/CD3c heterodimeric bispecific antibody. Methods are described in Example 8. The x axis shows the time (days) elapsed since tumor cells were implanted in the mice. The y axis shows the tumor volume (mm³). Downward pointing arrows over the x axis indicate the times at which the anti-MSLN/CD3c heterodimeric bispecific antibody, the control bispecific antibody, or Dulbecco's phosphate buffered saline (DPBS) was administered to the mice. Upward pointing arrows under the x axis indicate the times at which the anti-MSLN IgG1 antibody was administered. Symbols signify as follows: DPBS, open circles; P56019.5 (an anti-MSLN, anti-CD3 heterodimeric bispecific antibody), solidly filled squares; control bispecific antibody (anti-human EGFRviii/anti-human CD3), solidly filled triangles; anti-human MSLN IgG1, solidly filled diamonds; and NSG control mice, solidly filled circles.

FIG. 12: Intravenous pharmacokinetic properties of a heterodimeric bispecific antibody and a single chain bispecific molecule. Methods are explained in Example 9. The x axis shows the time (hours) post injection of the antibodies, and the y axis shows the serum concentration of the antibodies (ng/mL). The filled circles connected by solid lines denote data from the injection of the single chain bispecific antibody. The filed diamonds connected by solid lines denote data from the injection of the heterodimeric bispecific antibody.

FIG. 13: Subcutaneous pharmacokinetic properties of a heterodimeric bispecific antibody. The x axis shows the time (hours) post injection of the antibodies, and the y axis shows the serum concentration of the antibodies (ng/mL). Symbols are as in FIG. 11.

Brief Description of the Sequences

| SEQ ID NO | Description |
|---|---|
| SEQ ID NO: 1 | Amino acid sequence of human fibronectin 3 domain |
| SEQ ID NO: 2 | Amino acid sequence of human IgG1 Fc region |
| SEQ ID NO: 3 | Amino acid sequence of human IgG2 Fc region |
| SEQ ID NO: 4 | Amino acid sequence of human IgG3 Fc region |
| SEQ ID NO: 5 | Amino acid sequence of human IgG4 Fc region |
| SEQ ID NO: 6 | Amino acid sequence of the first polypeptide chain of P57216.9 |
| SEQ ID NO: 7 | Amino acid sequence of the second polypeptide chain of P57216.9 |
| SEQ ID NO: 8 | Amino acid sequence of the first polypeptide chain of P56019.5 |
| SEQ ID NO: 9 | Amino acid sequence of the second polypeptide chain of P56019.5 |
| SEQ ID NQ: 10 | Amino acid sequence of the first polypeptide chain of H71362.2 |
| SEQ ID NO: 11 | Amino acid sequence of the second polypeptide chain of H71362.2 |
| SEQ ID NO: 12 | Amino acid sequence of the first polypeptide chain of P69058.3 |
| SEQ ID NO: 13 | Amino acid sequence of the second polypeptide chain of P69058.3 |
| SEQ ID NO: 14 | Amino acid sequence of the first polypeptide chain of P69059.3 |
| SEQ ID NO: 15 | Amino acid sequence of the second polypeptide chain of P69059.3 |
| SEQ ID NO: 16 | Amino acid sequence of the first polypeptide chain of E73356.3 |
| SEQ ID NO: 17 | Amino acid sequence of the second polypeptide chain of E73356.3 |
| SEQ ID NO: 18 | Amino acid sequence of the first polypeptide chain of E73352.3 |
| SEQ ID NO: 19 | Amino acid sequence of the second polypeptide chain of E73352.3 |
| SEQ ID NO: 20 | Amino acid sequence of the first polypeptide chain of P136797.3 |
| SEQ ID NO: 21 | Amino acid sequence of the second polypeptide chain of P136797.3 |
| SEQ ID NO: 22 | Amino acid sequence of the first polypeptide chain of P136795.3 |
| SEQ ID NO: 23 | Amino acid sequence of the second polypeptide chain of P136795.3 |
| SEQ ID NO: 24 | Amino acid sequence of the first polypeptide chain of H69070.4 |
| SEQ ID NO: 25 | Amino acid sequence of the second polypeptide chain of H69070.4 |
| SEQ ID NO: 26 | Amino acid sequence of the first polypeptide chain of H69071.4 |
| SEQ ID NO: 27 | Amino acid sequence of the second polypeptide chain of H69071.4 |
| SEQ ID NO: 28 | Amino acid sequence of the first polypeptide chain of H69072.4 |
| SEQ ID NO: 29 | Amino acid sequence of the second polypeptide chain of H69072.4 |
| SEQ ID NO: 30 | Amino acid sequence of the first polypeptide chain of H71365.2 |
| SEQ ID NO: 31 | Amino acid sequence of the second polypeptide chain of H71365.2 |
| SEQ ID NO: 32 | Polynucleotide sequence encoding first polypeptide chain of P57216.9 |
| SEQ ID NO: 33 | Polynucleotide sequence encoding second polypeptide chain of P57216.9 |
| SEQ ID NO: 34 | Polynucleotide sequence encoding first polypeptide chain of P69058.3 |
| SEQ ID NO: 35 | Polynucleotide sequence encoding second polypeptide chain of P69058.3 |
| SEQ ID NO: 36 | Polynucleotide sequence encoding first polypeptide chain of P69059.3 |
| SEQ ID NO: 37 | Polynucleotide sequence encoding second polypeptide chain of P69059.3 |
| SEQ ID NO: 38 | Polynucleotide sequence encoding first polypeptide chain of P136795.3 |
| SEQ ID NO: 39 | Polynucleotide sequence encoding second polypeptide chain of P136795.3 |
| SEQ ID NO: 40 | Mature amino acid sequence of CD3 epsilon chain of Homo sapiens |
| SEQ ID NO: 41 | Mature amino acid sequence of CD3 epsilon chain of Macaca fascicularis |
| SEQ ID NO: 42 | Amino acid sequence of anti-CD3ε VH region (8H9) |
| SEQ ID NO: 43 | Amino acid sequence of anti-CD3ε VL region (901) |
| SEQ ID NO: 44 | Amino acid sequence of anti-CD3ε VH region (F12Q) |
| SEQ ID NO: 45 | Amino acid sequence of anti-CD3ε VL region (F12Q) |
| SEQ ID NO: 46 | Amino acid sequence of the first immunoglobulin variable region of P69058.3 |
| SEQ ID NO: 47 | Amino acid sequence of the third immunoglobulin variable region of P69058.3 |
| SEQ ID NO: 48 | Amino acid sequence of the fourth immunoglobulin variable region of P69058.3 |
| SEQ ID NO: 49 | Amino acid sequence of the second immunoglobulin variable region of P69059.3 |
| SEQ ID NO: 42 | Amino acid sequence of the fourth immunoglobulin variable region of P69059.3 |
| SEQ ID NO: 50 | Amino acid sequence of the first immunoglobulin variable region of H69072.4 |
| SEQ ID NO: 51 | Amino acid sequence of the fourth immunoglobulin variable region of H69072.4 |
| SEQ ID NO: 52 | Amino acid sequence of the second immunoglobulin variable region of P136795.3 |
| SEQ ID NO: 53 | Amino acid sequence of the third immunoglobulin variable region of P136795.3 |
| SEQ ID NO: 54 | Amino acid sequence of a peptide insertion that increases half life |
| SEQ ID NO: 55 | Amino acid sequence of a peptide insertion that increases half life |
| SEQ ID NO: 56 | Amino acid sequence of a peptide insertion that increases half life |
| SEQ ID NO: 57 | Amino acid sequence of a peptide insertion that increases half life |
| SEQ ID NO: 58 | Amino acid sequence of a peptide insertion that increases half life |
| SEQ ID NO: 59 | Amino acid sequence of a peptide insertion that increases half life |
| SEQ ID NO: 60 | Amino acid sequence of a peptide insertion that increases half life |
| SEQ ID NO: 61 | Amino acid sequence of a peptide insertion that increases half life |
| SEQ ID NO: 62 | Amino acid sequence of a peptide insertion that increases half life |
| SEQ ID NO: 63 | Amino acid sequence of a peptide insertion that increases half life |
| SEQ ID NO: 64 | Amino acid sequence of a peptide insertion that increases half life |
| SEQ ID NO: 65 | Amino acid sequence of a peptide insertion that increases half life |
| SEQ ID NO: 66 | Amino acid sequence of a linker |
| SEQ ID NO: 67 | Amino acid sequence of a linker |
| SEQ ID NO: 68 | Amino acid sequence of a linker |
| SEQ ID NO: 69 | Amino acid sequence of a linker |
| SEQ ID NO: 70 | Amino acid sequence of a CH1 region |
| SEQ ID NO: 71 | Amino acid sequence of CL region |
| SEQ ID NO: 72 | Amino acid sequence of VL specific to MSLN |
| SEQ ID NO: 73 | Amino acid sequence of CL region |
| SEQ ID NO: 74 | Amino acid sequence of a linker |
| SEQ ID NO: 75 | Amino acid sequence of an anti-HER2/CD3 single chain bispecific molecule |
| SEQ ID NO: 76 | Amino acid sequence of an anti-FOLR1/CD3 single chain bispecific molecule |
| SEQ ID NO: 77 | Amino acid sequence preceding a heavy chain CDR1 |
| SEQ ID NO: 78 | Amino acid preceding a heavy chain CDR2 |
| SEQ ID NO: 79 | Amino acid sequence following a heavy chain CDR3 |
| SEQ ID NO: 80 | Amino acid sequence preceding a light chain CDR3 |
| SEQ ID NO: 81 | Amino acid sequence of a portion of an epitope on CD3ε |
| SEQ ID NO: 82 | Amino acid sequence of an anti-CD3e VH region (12C) |
| SEQ ID NO: 83 | Amino acid sequence of an anti-CD3e VL region (12C) |

### DETAILED DESCRIPTION

Described herein is a new form of bispecific antibody. It is a heterodimeric molecule containing two different polypeptide chains, each comprising two immunoglobulin variable regions and, optionally, either a CH1 domain or a CK or CX domain. Together, the two chains contain two different binding sites, each of which comprises a heavy and light chain immunoglobulin variable (VH and VL) region and each of which binds to a different protein. In some embodiments, one of the proteins is expressed on the surface of an immune effector cell, such as a T cell, an NK cell, a macrophage, or a neutrophil and the other protein is expressed on the surface of a target cell, for example a cancer cell, a cell infected by a pathogen such as a virus, or a cell that mediates a fibrotic, autoimmune, or inflammatory disease. Since a heterodimeric bispecific antibody, as described herein, has only one binding site for each of the proteins it binds to (Le., it binds “monovalently” to each protein), its binding will not oligomerize the proteins it binds to on a cell surface. For example, if it binds to CD3 on the surface of a T cell, CD3 will not be oligomerized on the T cell surface. Oligomerization of CD3 can cause a generalized activation of a T cell, which can be undesirable. The heterodimeric bispecific antibody described herein tethers an immune effector cell to a target cell to, forming a close physical association between the cells and thereby eliciting a specific cytolytic activity against the target cell, rather than a generalized inflammatory response. The mechanism of action may be similar to that explored in detail for other bispecific antibodies. See, e.g., Haas etas. (2009), Immunobiology 214(6): 441-453. Further, the heterodimeric bispecific antibodies comprise at least one, optionally two, half life-extending moieties. Thus, they have favorable pharmacokinetic properties and are not unduly complex to manufacture since they contain only two different polypeptide chains.

### Definitions

An “antibody,” as meant herein, is a protein containing at least one VH or VL region, in many cases a heavy and a light chain variable region. Thus, the term “antibody” encompasses molecules having a variety of formats, including single chain Fv antibodies (scFv, which contain VH and VL regions joined by a linker), Fab, F(ab)₂′, Fab', scFv:Fc antibodies (as described in Carayannopoulos and Capra, Ch. 9 in FUNDAMENTAL IMMUNOLOGY, 3^{rd} ed., Paul, ed., Raven Press, New York, 1993, pp. 284-286) or full length antibodies containing two full length heavy and two full length light chains, such as naturally-occurring IgG antibodies found in mammals. Id. Such IgG antibodies can be of the IgG1, IgG2, IgG3, or IgG4 isotype and can be human antibodies. The portions of Carayannopoulos and Capra that describe the structure of antibodies are incorporated herein by reference. Further, the term “antibody” includes dimeric antibodies containing two heavy chains and no light chains such as the naturally-occurring antibodies found in camels and other dromedary species and sharks. See, e.g., Muldermans et al., 2001, J. Biotechnol. 74:277-302; Desmyter et at, 2001, J. Biol. Chem. 276:26285-90; Streltsov et at (2005), Protein Science 14: 2901-2909. An antibody can be “monospecific” (that is, binding to only one kind of antigen), “bispecific” (that is, binding to two different antigens), or “multispecific” (that is, binding to more than one different antigen). Further, an antibody can be monovalent, bivalent, or multivalent, meaning that it can bind to one, two, or multiple antigen molecules at once, respectively. An antibody binds “monovalently” to a particular protein when one molecule of the antibody binds to only one molecule of the protein, even though the antibody may also bind to a different protein as well. That is, an antibody binds “monovalently,” as meant herein, to two different proteins when it binds to only one molecule of each protein. Such an antibody is “bispecific” and binds to each of two different proteins “monovalently.” An antibody can be “monomeric,” Le., comprising a single polypeptide chain. An antibody can comprise multiple polypeptide chains (“multimeric”) or can comprise two (“dimeric”), three (“trimeric”), or four (“tetrameric”) polypeptide chains. If multimeric, an antibody can be a homomulitmer, i.e., containing more than one molecule of only one kind of polypeptide chain, including homodimers, homotrimer, or homotetramers. Alternatively, a multimeric antibody can be a heteromultimer, i.e., containing more than one different kind of polypeptide chain, including heterodimers, heterotrimers, or heterotetramers. An antibody can have a variety of possible formats including, for example, monospecific monovalent antibodies (as described in International Application WO 2009/089004 and US Publication 2007/0105199, the relevant portions of which are incorporated herein by reference) that may inhibit or activate the molecule to which they bind, bivalent monospecific or bispecific dimeric Fv-Fc, scFv-Fc, or diabody Fc, monospecific monovalent scFv-Fc/Fc's, the multispecific binding proteins and dual variable domain immunoglobulins described in US Publication 2009/0311253 (the relevant portions of which are incorporated herein by reference), the heterodimeric bispecific antibodies described herein, and the many formats for bispecific antibodies described in Chapters 1, 4, 5, 6, 7, 8, 9, 10, 11, 12, 13, 14 of BISPECIFIC ANTIBODIES, Kontermann, ed., Springer, 2011 (which chapters are incorporated herein by reference), among many other possible antibody formats.

A “cancer cell antigen,” as meant herein, is a protein expressed on the surface of a cancer cell. Some cancer cell antigens are also expressed on some normal cells, and some are specific to cancer cells. Cancer cell antigens can be highly expressed on the surface of a cancer cell. There are a wide variety of cancer cell antigens. Examples of cancer cell antigens include, without limitation, the following human proteins: epidermal growth factor receptor (EGFR), EGFRvIII (a mutant form of EGFR), melanoma-associated chondroitin sulfate proteoglycan (MCSP), mesothelin (MSLN), folate receptor 1 (FOLR1), and human epidermal growth factor 2 (HER2), among many others.

“Chemotherapy,” as used herein, means the treatment of a cancer patient with a “chemotherapeutic agent” that has cytotoxic or cytostatic effects on cancer cells. A “chemotherapeutic agent” specifically targets cells engaged in cell division and not cells that are not engaged in cell division. Chemotherapeutic agents directly interfere with processes that are intimately tied to cell division such as, for example, DNA replication, RNA synthesis, protein synthesis, the assembly, disassembly, or function of the mitotic spindle, and/or the synthesis or stability of molecules that play a role in these processes, such as nucleotides or amino acids. A chemotherapeutic agent therefore has cytotoxic or cytostatic effects on both cancer cells and other cells that are engaged in cell division. Chemotherapeutic agents are well-known in the art and include, for example: alkylating agents (e.g. busulfan, temozolomide, cyclophosphamide, lomustine (CCNU), methyllomustine, streptozotocin, ds-diamminedi-chloroplatinum, aziridinylbenzo-quinone, and thiotepa); inorganic ions (e.g. cisplatin and carboplatin); nitrogen mustards (e.g. melphalan hydrochloride, ifosfamide, chlorambucil, and mechlorethamine HCl); nitrosoureas (e.g. carmustine (BCNU)); anti-neoplastic antibiotics (e.g. adriamycin (doxorubicin), daunomycin, mitomycin C, daunorubicin, idarubicin, mithramycin, and bleomycin); plant derivatives (e.g. vincristine, vinblastine, vinorelbine, paclitaxel, docetaxel, vindesine, VP-16, and VM-26); antimetabolites (e.g. methotrexate with or without leucovorin, 5-fluorouracil with or without leucovorin, 5-fluorodeoxyuridine, 6-mercaptopurine, 6-thioguanine, cytarabine, 5-azacytidine, hydroxyurea, deoxycoformycin, gemcitabine, and fludarabine); podophyllotoxins (e.g. etoposide, irinotecan, and topotecan); as well as actinomycin D, dacarbazine (DTIC), mAMSA, procarbazine, hexamethylmelamine, pentamethylmelamine, L-asparaginase, and mitoxantrone, among many known in the art. See e.g. Cancer: Principles and Practice of Oncology, 4^{th} Edition, DeVita et al., eds., J.B. Lippincott Co., Philadelphia, Pa. (1993), the relevant portions of which are incorporated herein by reference. Alkylating agents and nitrogen mustard act by alkylating DNA, which restricts uncoiling and replication of strands. Methotrexate, cytarabine, 6-mercaptopurine, 5-fluorouracil, and gemcitabine interfere with nucleotide synthesis. Plant derivatives such a paclitaxel and vinblastine are mitotic spindle poisons. The podophyllotoxins inhibit topoisomerases, thus interfering with DNA replication. Antibiotics doxorubicin, bleomycin, and mitomycin interfere with DNA synthesis by intercalating between the bases of DNA (inhibiting uncoiling), causing strand breakage, and alkylating DNA, respectively. Other mechanisms of action include carbamoylation of amino acids (lomustine, carmustine), and depletion of asparagine pools (asparaginase). Merck Manual of Diagnosis and Therapy, 17^{th} Edition, Section 11, Hematology and Oncology, 144. Principles of Cancer Therapy, Table 144-2 (1999). Specifically included among chemotherapeutic agents are those that directly affect the same cellular processes that are directly affected by the chemotherapeutic agents listed above.

A drug or treatment is “concurrently” administered with a heterodimeric bispecific antibody, as meant herein, if it is administered in the same general time frame as the antibody, optionally, on an ongoing basis. For example, if a patient is taking Drug A once a week on an ongoing basis and the antibody once every six months on an ongoing basis, Drug A and the antibody are concurrently administered, whether or not they are ever administered on the same day. Similarly, if the antibody is taken once per week on an ongoing basis and Drug A is administered only once or a few times on a daily basis, Drug A and the antibody are concurrently administered as meant herein. Similarly, if both Drug A and the antibody are administered for short periods of time either once or multiple times within a one month period, they are administered concurrently as meant herein as long as both drugs are administered within the same month.

A “conservative amino acid substitution,” as meant herein, is a substitution of an amino acid with another amino acid with similar properties. Properties considered include chemical properties such as charge and hydrophobicity. Table 1 below lists substitutions for each amino acid that are considered to be conservative substitutions as meant herein.

TABLE 1
Conservative Amino Acid Substitutions

| Original Residue | Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala, Phe, Norleucine |
| Leu | Norleucine, Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala, Norleucine |

As meant herein, an “Fc region” is a dimer consisting of two polypeptide chains joined by one or more disulfide bonds, each chain comprising part or all of a hinge domain plus a CH2 and a CH3 domain. Each of the polypeptide chains is referred to as an “Fc polypeptide chain.” To distinguish the two Fc polypeptide chains, in some instances one is referred to herein as an “A chain” and the other is referred to as a “B chain.” More specifically, the Fc regions contemplated for use with the present invention are IgG Fc regions, which can be mammalian, for example human, IgG1, IgG2, IgG3, or IgG4 Fc regions. Among human IgG1 Fc regions, at least two allelic types are known. In other embodiments, the amino acid sequences of the two Fc polypeptide chains can vary from those of a mammalian Fc polypeptide by no more than 10 substitutions, insertions, and/or deletions of a single amino acid per 100 amino acids of sequence relative to a mammalian Fc polypeptide amino acid sequence. In some embodiments, such variations can be “heterodimerizing alterations” that facilitate the formation of heterodimers over homodimers, an Fc alteration that extends half life, an alteration that inhibits Fc gamma receptor (FcγR) binding, and/or an alteration that enhances ADCC.

An “Fc alteration that extends half life,” as meant herein is an alteration within an Fc polypeptide chain that lengthens the in vivo half life of a protein that contains the altered Fc polypeptide chain as compared to the half life of a similar protein containing the same Fc polypeptide, except that it does not contain the alteration. Such alterations can be included in an Fc polypeptide chain that is part of a heterodimeric bispecific antibody as described herein. The alterations M252Y, S254T, and T256E (methionine at position 252 changed to tyrosine; serine at position 254 changed to threonine; and threonine at position 256 changed to glutamic acid; numbering according to EU numbering as shown in Table 2) are Fc alterations that extend half life and can be used together, separately or in any combination. These alterations and a number of others are described in detail in U.S. Pat. No. 7,083,784. The portions of U.S. Patent 7,083,784 that describe such alterations are incorporated herein by reference. Similarly, M428L and N434S are Fc alterations that extend half life and can be used together, separately or in any combination. These alterations and a number of others are described in detail in U.S. Patent Application Publication 2010/0234575 and U.S. Pat. No. 7,670,600. The portions of U.S. Patent Application Publication 2010/0234575 and U.S. Pat. No. 7,670,600 that describe such alterations are incorporated herein by reference. In addition, any substitution at one of the following sites can be considered an Fc alteration that extends half life as meant here: 250, 251, 252, 259, 307, 308, 332, 378, 380, 428, 430, 434, 436. Each of these alterations or combinations of these alterations can be used to extend the half life of a heterodimeric bispecific antibody as described herein. Other alterations that can be used to extend half life are described in detail in International Application PCT/US2012/070146 filed December 17, 2012. The portions of this application that describe such alterations are incorporated herein by reference. Some specific embodiments described in this application include insertions between positions 384 and 385 (EU numbering as shown in Table 2) that extend half life, including the following amino acid sequences: GGCVFNMFNCGG (SEQ ID NO:54), GGCHLPFAVCGG (SEQ ID NO:55), GGCGHEYMWCGG (SEQ ID NO:56), GGCWPLQDYCGG(SEQ ID NO:57), GGCMQMNKWCGG (SEQ ID NO:58), GGCDGRTKYCGG (SEQ ID NO:59), GGCALYPTNCGG (SEQ ID NO:60), GGCGKHWHQCGG (SEQ ID NO:61), GGCHSFKHFCGG (SEQ ID NO:62), GGCQGMWTWCGG (SEQ ID NO:63), GGCAQQWHHEYCGG (SEQ ID NO:64), and GGCERFHHACGG (SEQ ID NO:65), among others. Heterodimeric bispecific antibodies containing such insertions are contemplated.

A “half life-extending moiety,” as meant herein, is a molecule that extends the in vivo half life of a protein to which it is attached as compared to the in vivo half life of the protein without the half life-extending moiety. Methods for measuring half life are well known in the art. A method for ascertaining half life is disclosed in Example 9. A half life-extending moiety can be a polypeptide, for example an Fc polypeptide chain or a polypeptide that can bind to albumin. The amino acid sequence of a domain of human fibronectin type III (Fn3) that has been engineered to bind to albumin is provided in SEQ ID NO:1, and various human IgG Fc polypeptide sequences are given in SEQ ID NOs:2-5. In alternate embodiments, a half life-extending moiety can be a non-polypeptide molecule. For example, a polyethylene glycol (PEG) molecule can be a half life-extending moiety.

“Heterodimerizing alterations” generally refer to alterations in the A and B chains of an Fc region that facilitate the formation of heterodimeric Fc regions, that is, Fc regions in which the A chain and the B chain of the Fc region do not have identical amino acid sequences. Such alterations can be included in an Fc polypeptide chain that is part of a heterodimeric bispecific antibody as described herein. Heterodimerizing alterations can be asymmetric, that is, a A chain having a certain alteration can pair with a B chain having a different alteration. These alterations facilitate heterodimerization and disfavor homodimerization. Whether hetero- or homo-dimers have formed can be assessed by size differences as determined by polyacrylamide gel electrophoresis in some situations or by other appropriate means such as differing charges or biophysical characteristics, including binding by antibodies or other molecules that recognize certain portions of the heterodimer including molecular tags. One example of such paired heterodimerizing alterations are the so-called “knobs and holes” substitutions. See, e.g., U.S. Pat. No. 7,695,936 and US Patent Application Publication 2003/0078385, the portions of which describe such mutations are incorporated herein by reference. As meant herein, an Fc region that contains one pair of knobs and holes substitutions, contains one substitution in the A chain and another in the B chain. For example, the following knobs and holes substitutions in the A and B chains of an IgG1 Fc region have been found to increase heterodimer formation as compared with that found with unmodified A and B chains: 1) Y4071 in one chain and T366Y in the other; 2) Y407A in one chain and T366W in the other; 3) F405A in one chain and T394W in the other; 4) F405W in one chain and T394S in the other; 5) Y4071 in one chain and T366Y in the other; 6) T366Y and F405A in one chain and T394W and Y4071 in the other; 7) T366W and F405W in one chain and T394S and Y407A in the other; 8) F405W and Y407A in one chain and T366W and T394S in the other; and 9) T366W in one polypeptide of the Fc and T366S, L368A, and Y407V in the other. This way of notating mutations can be explained as follows. The amino acid (using the one letter code) normally present at a given position in the CH3 region using the EU numbering system (which is presented in Edelman et al. (1969), Proc. Natl. Acad. Sci. 63: 78-85; see also Table 2 below) is followed by the EU position, which is followed by the alternate amino acid that is present at that position. For example, Y4071 means that the tyrosine normally present at EU position 407 is replaced by a threonine. Alternatively or in addition to such alterations, substitutions creating new disulfide bridges can facilitate heterodimer formation. See, e.g., US Patent Application Publication 2003/0078385, the portions of which describe such mutations are incorporated herein by reference. Such alterations in an IgG1 Fc region include, for example, the following substitutions: Y349C in one Fc polypeptide chain and 5354C in the other; Y349C in one Fc polypeptide chain and E356C in the other; Y349C in one Fc polypeptide chain and E357C in the other; L351C in one Fc polypeptide chain and 5354C in the other; T394C in one Fc polypeptide chain and E397C in the other; or D399C in one Fc polypeptide chain and K392C in the other. Similarly, substitutions changing the charge of a one or more residue, for example, in the C_{H}3-C_{H}3 interface, can enhance heterodimer formation as explained in WO 2009/089004, the portions of which describe such substitutions are incorporated herein by reference. Such substitutions are referred to herein as “charge pair substitutions,” and an Fc region containing one pair of charge pair substitutions contains one substitution in the A chain and a different substitution in the B chain. General examples of charge pair substitutions include the following: 1) K409D or K409E in one chain plus D399K or D399R in the other; 2) K392D or K392E in one chain plus D399K or D399R in the other; 3) K439D or K439E in one chain plus E356K or E356R in the other; and 4) K370D or K370E in one chain plus E357K or E357R in the other. In addition, the substitutions R355D, R355E, K360D, or K360R in both chains can stabilize heterodimers when used with other heterodimerizing alterations. Specific charge pair substitutions can be used either alone or with other charge pair substitutions.

Specific examples of single pairs of charge pair substitutions and combinations thereof include the following: 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; and 17) K409D and K439D on one chain plus D399K and E356K on the other. Any of the these heterodimerizing alterations can be used in the Fc regions of the heterodimeric bispecific antibodies described herein.

An “alteration that inhibits FcγR binding,” as meant herein, is one or more insertions, deletions, or substitutions within an Fc polypeptide chain that inhibits the binding of FcγRIIA, FcγRIIB, and/or FcγRIIIA as measured, for example, by an ALPHALISA®-based competition binding assay (PerkinElmer, Waltham, Mass.). Such alterations can be included in an Fc polypeptide chain that is part of a heterodimeric bispecific antibody as described herein. More specifically, alterations that inhibit Fc gamma receptor (FcγR) binding include L234A, L235A, or any alteration that inhibits glycosylation at N297, including any substitution at N297. In addition, along with alterations that inhibit glycosylation at N297, additional alterations that stabilize a dimeric Fc region by creating additional disulfide bridges are also contemplated. Further examples of alterations that inhibit FcγR binding include a D265A alteration in one Fc polypeptide chain and an A327Q alteration in the other Fc polypeptide chain.

An “alteration that enhances ADCC,” as meant herein is one or more insertions, deletions, or substitutions within an Fc polypeptide chain that enhances antibody dependent cell-mediated cytotoxicity (ADCC). Such alterations can be included in an Fc polypeptide chain that is part of a heterodimeric bispecific antibody as described herein. Many such alterations are described in International Patent Application Publication WO 2012/125850. Portions of this application that describe such alterations are incorporated herein by reference. Such alterations can be included in an Fc polypeptide chain that is part of a heterodimeric bispecific antibody as described herein. ADCC assays can be performed as follows. Cell lines that express high and lower amounts of a cancer cell antigen on the cell surface can be used as target cells. These target cells can belabeled with carboxyfluorescein succinimidyl ester (CFSE) and then washed once with phosphate buffered saline (PBS) before being deposited into 96-well microtiter plates with V-shaped wells. Purified immune effector cells, for example T cells or NK cells, can be added to each well. A monospecific antibody that binds to the cancer antigen and contains the alteration(s) being tested and an isotype-matched control antibody can be diluted in a 1:3 series and added to the wells. The cells can be incubated at 37° C. with 5% CO₂ for 3.5 hrs. The cells can be spun down and re-suspended in lx FACS buffer (1× phosphate buffered saline (PBS) containing 0.5% fetal bovine serum (FBS)) with the dye TO-PRO®-3 iodide (Molecular Probes, Inc. Corporation, Oregon, USA), which stains dead cells, before analysis by fluorescence activated cell sorting (FACS). The percentage of cell killing can be calculated using the following formula:

(percent tumor cell lysis with bispecific−percent tumor cell lysis without bispecific)/(percent total cell lysis−percent tumor cell lysis without bispecific)

Total cell lysis is determined by lysing samples containing effector cells and labeled target cells without a bispecific molecule with cold 80% methanol. Exemplary alterations that enhance ADCC include the following alterations in the A and B chains of anFc region: (a) the A chain comprises Q311M and K334V substitutions and the B chain comprises L234Y, E294L, and Y296W substitutions or vice versa; (b) the A chain comprises E233L, Q311M, and K334V substitutions and the B chain comprises L234Y, E294L, and Y296W substitutions or vice versa; (c) the A chain comprises L234I, Q311M, and K334V substitutions and the B chain comprises L234Y, E294L, and Y296W substitutions or vice versa; (d) the A chain comprises S298T and K334V substitutions and the B chain comprises L234Y, K290Y, and Y296W substitutions or vice versa; (e) the A chain comprises A330M and K334V substitutions and the B chain comprises L234Y, K290Y, and Y296W substitutions or vice versa; (f) the A chain comprises A330F and K334V substitutions and the B chain comprises L234Y, K290Y, and Y296W substitutions or vice versa; (g) the A chain comprises Q311M, A330M, and K334V substitutions and the B chain comprises L234Y, E294L, and Y296W substitutions or vice versa; (h) the A chain comprises Q311M, A330F, and K334V substitutions and the B chain comprises L234Y, E294L, and Y296W substitutions or vice versa; (i) the A chain comprises S298T, A330M, and K334V substitutions and the B chain comprises L234Y, K290Y, and Y296W substitutions or vice versa; (j) the A chain comprises S298T, A330F, and K334V substitutions and the B chain comprises L234Y, K290Y, and Y296W substitutions or vice versa; (k) the A chain comprises S239D, A330M, and K334V substitutions and the B chain comprises L234Y, K290Y, and Y296W substitutions or vice versa; (l) the A chain comprises S239D, S298T, and K334V substitutions and the B chain comprises L234Y, K290Y, and Y296W substitutions or vice versa; (m) the A chain comprises a K334V substitution and the B chain comprises Y296W and S298C substitutions or vice versa; (n) the A chain comprises a K334V substitution and the B chain comprises L234Y, Y296W, and S298C substitutions or vice versa; (o) the A chain comprises L235S, S239D, and K334V substitutions and the B chain comprises L234Y, K290Y, and Y296W, substitutions or vice versa; (p) the A chain comprises L235S, S239D, and K334V substitutions and the B chain comprises L234Y, Y296W, and S298C substitutions or vice versa; (q) the A chain comprises Q311M and K334V substitutions and the B chain comprises L234Y, F243V, and Y296W substitutions or vice versa; (r) the A chain comprises Q311M and K334V substitutions and the B chain comprises L234Y, K296W, and S298C substitutions or vice versa; (s) the A chain comprises S239D and K334V substitutions and the B chain comprises L234Y, K290Y, and Y296W substitutions or vice versa; (t) the A chain comprises S239D and K334V substitutions and the B chain comprises L234Y, Y296W, and S298C substitutions or vice versa; (u) the A chain comprises F243V and K334V substitutions and the B chain comprises L234Y, K290Y, and Y296W, substitutions or vice versa; (v) the A chain comprises F243V and K334V substitutions and the B chain comprises L234Y, Y296W, and S298C substitutions or vice versa; (w) the A chain comprises E294L and K334V substitutions and the B chain comprises L234Y, K290Y, and Y296W substitutions or vice versa; (x) the A chain comprises E294L and K334V substitutions and the B chain comprises L234Y, Y296W, and S298C substitutions or vice versa; (y) the A chain comprises A330M and K334V substitutions and the B chain comprises L234Y and Y296W substitutions or vice versa; or (z) the A chain comprises A330M and K334V substitutions and the B chain comprises K290Y and Y296W substitutions or vice versa.

An “IgG antibody,” as meant herein, is an antibody consisting essentially of two immunoglobulin IgG heavy chains and two immunoglobulin light chains, which can be kappa or lambda light chains. More specifically, the heavy chains contain a VH region, a CH1 region, a hinge region, a CH2 region, and a CH3 region, while the light chains contain a VL region and a CL region. Numerous sequences of such immunoglobulin regions are known in the art. See, e.g., Kabat et at in SEQUENCES OF IMMUNOLOGICAL INTEREST, Public Health Service N.I.H., Bethesda, Md., 1991. Sequences of regions from IgG antibodies disclosed in Kabat et al. are incorporated herein by reference.

An “immune effector cell,” as meant herein, is a cell that is involved in the mediation of a cytolytic immune response, including, for example, T cells, NK cells, macrophages, or neutrophils. The heterodimeric bispecific antibodies described herein bind to an antigen that is part of a protein expressed on the surface of an immune effector cell. Such proteins are referred to herein as “effector cell proteins.”

An “immunoglobulin heavy chain,” as meant herein, consists essentially of a VH region, a CH1 region, a hinge region, a CH2 region, a CH3 region in that order, and, optionally, a region downstream of the CH3 region in some isotypes. Close variants of an immunoglobulin heavy chain containing no more than 10 amino acid substitutions, insertions, and/or deletions of a single amino acid per 100 amino acids relative to a known or naturally occurring immunoglobulin heavy chain amino acid sequence are encompassed within what is meant by an immunoglobulin heavy chain.

A “immunoglobulin light chain,” as meant herein, consists essentially of a light chain variable region (VL) and a light chain constant domain (CL). Close variants of an immunoglobulin light chain containing no more than 10 amino acid substitutions, insertions, and/or deletions of a single amino acid per 100 amino acids relative to a known or naturally occurring immunoglobulin light chain amino acid sequence are encompassed within what is meant by an immunoglobulin light chain.

An “immunoglobulin variable region,” as meant herein, is a VH region, a VL region, or a variant thereof. Close variants of an immunoglobulin variable region containing no more than 10 amino acid substitutions, insertions, and/or deletions of a single amino acid per 100 amino acids relative to a known or naturally occurring immunoglobulin variable region amino acid sequence are encompassed within what is meant by an immunoglobulin variable region. Many examples of VH and VL regions are known in the art, such as, for example, those disclosed by Kabat et at in SEQUENCES OF IMMUNOLOGICAL INTEREST, Public Health Service N.I.H., Bethesda, Md., 1991. Based on the extensive sequence commonalities in the less variable portions of the VH and VL regions, the position within a sequence of more variable regions, and the predicted tertiary structure, one of skill in the art can recognize an immunoglobulin variable region by its sequence. See, e.g., Honegger and Plückthun (2001), J. Mol. Biol. 309: 657-670.

An immunoglobulin variable region contains three hypervariable regions, known as complementarity determining region 1 (CDR1), complementarity determining region 2 (CDR2), and complementarity determining region 3 (CDR3). These regions form the antigen binding site of an antibody. The CDRs are embedded within the less variable framework regions (FR1-FR4). The order of these subregions within an immunoglobulin variable region is as follows: FR1-CDR1-FR2-CDR2-FR3-CDR3-FR4. Numerous sequences of immunoglobulin variable regions are known in the art. See, e.g., Kabat et at, SEQUENCES OF PROTEINS OF IMMUNOLOGICAL INTEREST, Public Health Service N.I.H., Bethesda, Md., 1991.

CDRs can be located in a VH region sequence in the following way. CDR1 starts at approximately residue 31 of the mature VH region and is usually about 5-7 amino acids long, and it is almost always preceded by a Cys-Xxx-Xxx-Xxx-Xxx-Xxx-Xxx-Xxx-Xxx (SEQ ID NO:77) (where “Xxx” is any amino acid). The residue following the heavy chain CDR1 is almost always a tryptophan, often a Trp-Val, a Trp-Ile, or a Trp-Ala. Fourteen amino acids are almost always between the last residue in CDR1 and the first in CDR2, and CDR2 typically contains 16 to 19 amino acids. CDR2 may be immediately preceded by Leu-Glu-Trp-Ile-Gly (SEQ ID NO:78) and may be immediately followed by Lys/Arg-Leu/Ile/Val/Phe/Thr/Ala-Thr/Ser/Ile/Ala. Other amino acids may precede or follow CDR2. Thirty two amino acids are almost always between the last residue in CDR2 and the first in CDR3, and CDR3 can be from about 3 to 25 residues long. A Cys-Xxx-Xxx almost always immediately precedes CDR3, and a Trp-Gly-Xxx-Gly (SEQ ID NO: 79) almost always follows CDR3.

Light chain CDRs can be located in a VL region in the following way. CDR1 starts at approximately residue 24 of the mature antibody and is usually about 10 to 17 residues long. It is almost always preceded by a Cys. There are almost always 15 amino acids between the last residue of CDR1 and the first residue of CDR2, and CDR2 is almost always 7 residues long. CDR2 is typically preceded by Ile-Tyr, Val-Tyr, Ile-Lys, or Ile-Phe. There are almost always 32 residues between CDR2 and CDR3, and CDR3 is usually about 7 to 10 amino acids long. CDR3 is almost always preceded by Cys and usually followed by Phe-Gly-Xxx-Gly (SEQ ID NO:80).

A “linker,” as meant herein, is a peptide that links two polypeptides, which can be two immunoglobulin variable regions in the context of a heterodimeric bispecific antibody. A linker can be from 2-30 amino acids in length. In some embodiments, a linker can be 2-25, 2-20, or 3-18 amino acids long. In some embodiments, a linker can be a peptide no more than 14, 13, 12, 11, 10, 9, 8, 7, 6, or 5 amino acids long. In other embodiments, a linker can be 5-25, 5-15, 4-11, 10-20, or 20-30 amino acids long. In other embodiments, a linker can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long. Exemplary linkers include, for example, the amino acid sequences TVAAP (SEQ ID NO:66), ASTKGP (SEQ ID NO:67),GGGGSGGGGS (SEQ ID NO:68), GGGGSAAA (SEQ ID NO:69), GGGGSGGGGSGGGGS (SEQ ID NO:74), and AAA, among many others.

A heterodimeric bispecific antibody “mediates cytolysis of a target cell by an immune effector cell,” as meant herein, when addition of an amount from 0.001 pM to 20000 pM of the heterodimeric bispecific antibody to a cell cytolysis assay as described herein effectively elicits cytolysis of of the target cells.

“Non-chemotherapeutic anti-neoplastic agents” are chemical agents, compounds, or molecules having cytotoxic or cytostatic effects on cancer cells other than chemotherapeutic agents. Non-chemotherapeutic antineoplastic agents may, however, be targeted to interact directly with molecules that indirectly affect cell division such as cell surface receptors, including receptors for hormones or growth factors. However, non-chemotherapeutic antineoplastic agents do not interfere directly with processes that are intimately linked to cell division such as, for example, DNA replication, RNA synthesis, protein synthesis, or mitotic spindle function, assembly, or disassembly. Examples of non-chemotherapeutic anti-neoplastic agents include inhibitors of Bcl2, inhibitors of farnesyltransferase, anti-estrogenic agents such as tamoxifen, anti-androgenic compounds, interferon, arsenic, retinoic acid, retinoic acid derivatives, antibodies targeted to tumor-specific antigens, and inhibitors of the Bcr-Abl tyrosine kinase (e.g., the small molecule STI-571 marketed under the trade name GLEEVEC™ by Novartis, N.Y. and New Jersey, USA and Basel, Switzerland), among many possible non-chemotherapeutic anti-neoplastic agents.

A “target cell” is a cell that a heterodimeric bispecific antibody, as described herein, binds to and that is involved in mediating a disease. In some cases, a target cell can be a cell that is ordinarily involved in mediating an immune response, but is also involved in the mediation of a disease. For example in B cell lymphoma, a B cell, which is ordinarily involved in mediating immune response, can be a target cell. In some embodiments, a target cell is a cancer cell, a cell infected with a pathogen, or a cell involved in mediating an autoimmune or inflammatory disease. The heterodimeric bispecific antibody can bind to the target cell via binding to an antigen on a “target cell protein,” which is a protein that is displayed on the surface of the target cell, possibly a highly expressed protein.

“Tumor burden” refers to the number of viable cancer cells, the number of tumor sites, and/or the size of the tumor(s) in a patient suffering from a cancer. A reduction in tumor burden can be observed, for example, as a reduction in the amount of a tumor-associated antigen or protein in a patient's blood or urine, a reduction in the number of tumor cells or tumor sites, and/or a reduction in the size of one or more tumors.

A “therapeutically effective amount” of a heterodimeric bispecific antibody as described herein is an amount that has the effect of, for example, reducing or eliminating the tumor burden of a cancer patient or reducing or eliminating the symptoms of any disease condition that the protein is used to treat. A therapeutically effective amount need not completely eliminate all symptoms of the condition, but may reduce severity of one or more symptoms or delay the onset of more serious symptoms or a more serious disease that can occur with some frequency following the treated condition.

“Treatment” of any disease mentioned herein encompasses an alleviation of at least one symptom of the disease, a reduction in the severity of the disease, or the delay or prevention of disease progression to more serious symptoms that may, in some cases, accompany the disease or lead to at least one other disease. Treatment need not mean that the disease is totally cured. A useful therapeutic agent needs only to reduce the severity of a disease, reduce the severity of one or more symptoms associated with the disease or its treatment, or delay the onset of more serious symptoms or a more serious disease that can occur with some frequency following the treated condition.

When it is said that a named VH/VL pair of immunoglobulin variable regions can bind to a target cell or an immune effector cell “when they are part of an IgG antibody or scFv antibody,” it is meant that an IgG antibody that contains the named VH region in both heavy chains and the named VL region in both light chains or the scFv that contains the VH/VL pair can bind to the target cell or the immune effector cell. A binding assay is described in Example 2. One of skill in the art could construct an IgG or scFv antibody containing the desired sequences given the knowledge in the art.

### Heterodimeric Bispecific Antibodies

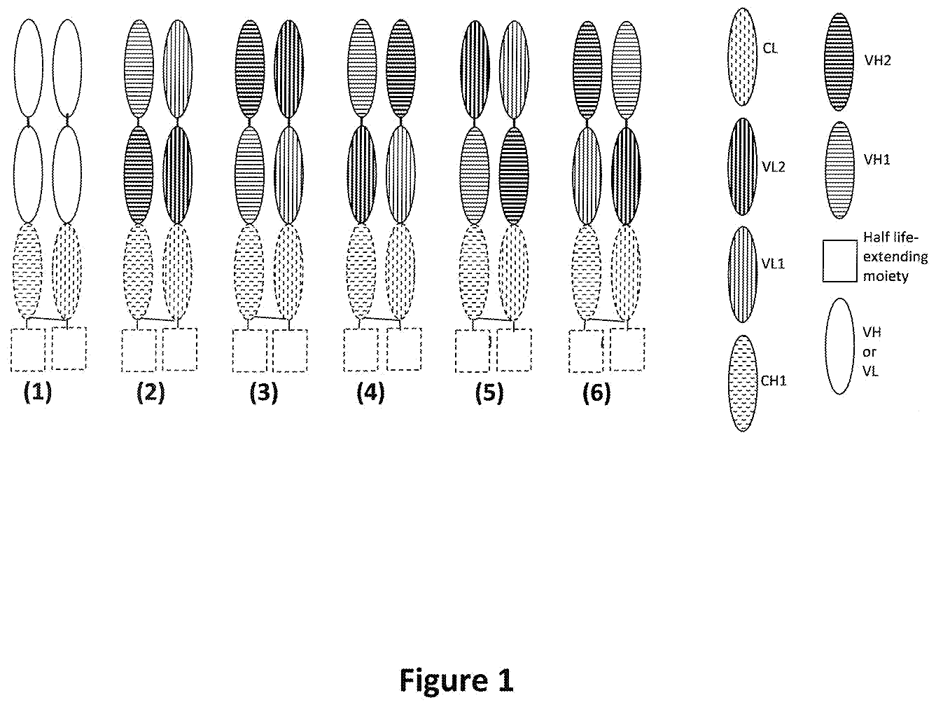

In the most general sense, a heterodimeric bispecific antibody as described herein comprises two polypeptide chains having different amino acid sequences, which, together, can bind to two different antigens. In addition, due to the inclusion of a half life-extending moiety, the heterodimeric bispecific antibodies have tunable pharmacokinetic properties, optionally including a half life between a few hours and a few days or from a few days to one or more weeks. In one embodiment, the first polypeptide chain comprises two immunoglobulin variable regions followed by a CH1 region, which is followed by a half-life extending moiety, and the second polypeptide chain comprises two immunoglobulin variable regions followed by a CL region. Optionally, the CL region can also be followed by a half life-extending moiety. This structure is illustrated in FIG. 1(1). In an alternate embodiment, the second polypeptide chain comprises two immunoglobulin variable regions followed by a CL region and then a half life-extending moiety, and the first polypeptide chain comprises two immunoglobulin variable regions followed by a CH1 region, which may or may not be followed by a half-life extending moiety. In some embodiments, the half-life extending moiety is an Fc polypeptide chain that is present on both the first and second polypeptide chains after the CH1 region and the CL region, respectively. In other embodiments, neither polypeptide chain includes a CH1 or a CL region, but at least one polypeptide chain includes a half life-extending moiety. In some such embodiments, both polypeptide chains include an Fc polypeptide chain.

More particular embodiments specify which immunoglobulin variable regions are VH or VL regions and which can associate to form a binding site for an antigen, which can be part of a protein expressed on the surface of an immune effector cell or a target cell. Generally, the antigen-binding portion of an antibody includes both a VH and a VL region, although in some cases a VH or a VL region can bind to an antigen without a partner. See, e.g., US Application Publication 2003/0114659. FIG. 1(2) illustrates an embodiment in which the two variable regions in what is referred as the first polypeptide chain (which contains a CH1 region) are two different VH regions, and the two variable regions in what is referred to as the second polypeptide chain (which contains a CL region) are two different VL regions. In this embodiment, the linkers between the two variable regions in both the first and second polypeptide chains are shorter than 12 amino acids. As a result, variable regions can pair “in parallel” to form the antigen binding sites. That is, the first VH region on the first polypeptide chain (VH1) can pair with the first VL region on the second polypeptide chain (VL1) to form a binding site for a first antigen. Further, the second VH region on the first polypeptide (VH2) can associate “in parallel” with the second VL region on the second polypeptide chain (VL2) to form a binding site for a second antigen binding site. The embodiment shown in FIG. 1(3) is similar except the order of the two VH regions and of the two VL regions is reversed, and the variable regions can also pair in parallel to form the antigen binding sites.

Other embodiments in which “in parallel” VH/VL interaction are required can have two VL regions on the first polypeptide chain and two VH regions on the second polypeptide chain. In another embodiment in which an “in parallel” interaction is required, the first polypeptide chain can comprise a VH region followed by a VL region and the second polypeptide chain can comprise a VL region followed by a VH region. Similarly, the first polypeptide chain could also comprise a VL region followed by a VH region, and the second polypeptide chain could comprise a VH region followed by a VL region.

FIG. 1(4) shows an embodiment in which the first variable region on the first polypeptide chain is the VH1 region, which is followed by the VL2 region. On the second polypeptide chain, the VH2 region is followed the VL1 region. In this format, the first variable region on the first polypeptide chain must associate with the second variable region on the second polypeptide chain to form a binding site for the first antigen. Similarly, the second variable region on the first polypeptide chain must associate with the first variable region on the second polypeptide chain to form a binding site for the second antigen. This situation is referred to herein as a “diagonal” interaction. Although the order of the variable regions on the first and second polypeptide chains in embodiments 1(5) and 1(6) is different, the variable regions in these embodiments must also pair in an diagonal interaction to form the antigen binding sites.

Between the two immunoglobulin variable regions on each polypeptide chain is a peptide linker, which can be the same on both polypeptide chains or different. The linkers can play a role in the structure of the antibody. If the linker is short enough, Le., less than 12 amino acids long, it will not allow enough flexibility for the two variable regions on a single polypeptide chain to interact to form an antigen binding site. Thus, short linkers make it more likely that a variable region will interact with a variable region on the other polypeptide chain to form an antigen binding site, rather than interacting with a variable region on the same polypeptide chain. If the linker is at least 15 amino acids long, it will allow a variable region to interact with another variable region on the same polypeptide chain to form an antigen binding site.

A half life-extending moiety can be, for example, an Fc polypeptide, albumin, an albumin fragment, a moiety that binds to albumin or to the neonatal Fc receptor (FcRn), a derivative of fibronectin that has been engineered to bind albumin or a fragment thereof, a peptide, a single domain protein fragment, or other polypeptide that can increase serum half life. In alternate embodiments, a half life-extending moiety can be a non-polypeptide molecule such as, for example, polyethylene glycol (PEG). Sequences of human IgG1, IgG2, IgG3, and IgG4 Fc polypeptides that could be used are provided in SEQ ID NOs:2-5. Variants of these sequences containing one or more heterodimerizing alterations, one or more Fc alteration that extends half life, one or more alteration that enhances ADCC, and/or one or more alteration that inhibits Fc gamma receptor (FcγR) binding are also contemplated, as are other close variants containing not more than 10 deletions, insertions, or substitutions of a single amino acid per 100 amino acids of sequence.

The sequence of a derivative of human fibronectin type III (Fn3) engineered to bind albumin is provided in SEQ ID NO:1. As is known in the art, the loops of a human fibronectin type III (Fn3) domain can be engineered to bind to other targets. Koide (1998), J Mol Biol: 284(4): 1141-51. Exemplary pairs of amino acid sequences that make up heterodimeric bispecific antibodies that contain an engineered fibronectin type III domain that can bind to albumin as a half life-extending moiety include the following: SEQ ID NOs:6 and 7; SEQ ID NOs:8 and 9; SEQ ID NOs:10 and 11; SEQ ID NO:s:12 and 13, and SEQ ID NOs:14 and 15.

The half life extending moiety can be an Fc region of an antibody. If so, the first polypeptide chain can contain an Fc polypeptide after the CH1 region, and the second polypeptide chain can contain an Fc polypeptide after the CL region. Alternatively, only one polypeptide chain can contain an Fc polypeptide chain. There can be, but need not be, a linker between the CH1 region and the Fc region and/or between the CL region and the Fc region. As explained above, an Fc polypeptide chain comprises all or part of a hinge region followed by a CH2 and a CH3 region. The Fc polypeptide chain can be of mammalian (for example, human, mouse, rat, rabbit, dromedary, or new or old world monkey), avian, or shark origin. In addition, as explained above, an Fc polypeptide chain can have a limited number alterations For example, an Fc polypeptide chain can comprise one or more heterodimerizing alterations, one or more alteration that inhibits binding to FcγR, or one or more alterations that increase binding to FcRn. Exemplary amino acid sequences of pairs of polypeptide chains that make up a heterodimeric bispecific antibody containing an Fc region include the following pairs of sequences: SEQ ID NOs:16 and 17; SEQ ID NOs:18 and 19; and SEQ ID NOs:20 and 21.

In some embodiments the amino acid sequences of the Fc polypeptides can be mammalian, for example a human, amino acid sequences. The isotype of the Fc polypeptide can be IgG, such as IgG1, IgG2, IgG3, or IgG4, IgA, IgD, IgE, or IgM. Table 2 below shows an alignment of the amino acid sequences of human IgG1, IgG2, IgG3, and IgG4 sequences.

TABLE 2
Amino acid sequences of human IgG Fc regions

| | |
|---|---|
| IgG1 | ----------------------------------------------- |
| IgG2 | ----------------------------------------------- |
| IgG3 | ELKTPLGDTTHTCPRCPEPKSCDTPPPCPRCPEPKSCDTPPPCPRCP |
| IgG4 | ----------------------------------------------- |
| | 225 235 245 255 265 275 |
| | * * * * * * |
| IgG1 | EPKSCDKTHTCPPCPAPELLGGPSVFLFPPKPKDTLMISRTPEVTCVVVDVSHEDPEVKE |
| IgG2 | ERKCCVE---CPPCPAPPVA-GPSVFLFPPKPKDTLMISRTPEVTCVVVDVSHEDPEVQF |
| IgG3 | EPKSCDTPPPCPRCPAPELLGGPSVFLFPPKPKDTLMISRTPEVTCVVVDVSHEDPEVQF |
| IgG4 | ESKYG---PPCPSCPAPEFLGGPSVFLFPPKPKDTLMISRTPEVTCVVVDVSQEDPEVQF |
| | 285 295 305 315 325 335 |
| | * * * * * * |
| IgG1 | NWYVDGVEVHNAKTKPREEQYNSTYRVVSVLTVLHQDWLNGKEYKCKVSNKALPAPIEKT |
| IgG2 | NWYVDGMEVHNAKTKPREEQFNSTFRVVSVLTVVHQDWLNGKEYKCKVSNKGLPAPIEKT |
| IgG3 | KWYVDGVEVHNAKTKPREEQYNSTFRVVSVLTVLHQDWLNGKEYKCKVSNKALPAPIEKT |
| IgG4 | NWYVDGVEVHNAKTKPREEQFNSTYRVVSVLTVLHQDWLNGKEYKCKVSNKGLPSSIEKT |
| | 345 355 365 375 385 395 |
| | * * * * * * |
| IgG1 | ISKAKGQPREPQVYTLPPSREEMTKNQVSLTCLVKGFYPSDIAVEWESNGQPENNYKTTP |
| IgG2 | ISKTKGQPREPQVYTLPPSREEMTKNQVSLTCLVKGFYPSDIAVEWESNGQPENNYKTTP |
| IgG3 | ISKTKGQPREPQVYTLPPSREEMTKNQVSLTCLVKGFYPSDIAVEWESSGQPENNYNTTP |
| IgG4 | ISKAKGQPREPQVYTLPPSQEEMTKNQVSLTCLVKGFYPSDIAVEWESNGQPENNYKTTP |
| | 405 415 425 435 445 |
| | * * * * * |
| IgG1 | PVLDSDGSFFLYSKLTVDKSRWQQGNVFSCSVMHEALHNHYTQKSLSLSPGK (SEQ ID NO: 2) |
| IgG2 | PMLDSDGSFFLYSKLTVDKSRWQQGNVFSCSVMHEALHNHYTQKSLSLSPGK (SEQ ID NO: 3) |
| IgG3 | PMLDSDGSFFLYSKLTVDKSRWQQGNIFSCSVMHEALHNRFTQKSLSLSPGK (SEQ ID NO: 4) |
| IgG4 | PVLDSDGSFFLYSRLTVDKSRWQEGNVFSCSVMHEALHNHYTQKSLSLSLGK (SEQ ID NO: 5) |

The numbering shown in Table 2 is according the EU system of numbering, which is based on the sequential numbering of the constant region of an IgG1 antibody. Edelman et al. (1969), Proc. Natl. Acad. Sci. 63: 78-85. Thus, it does not accommodate the additional length of the IgG3 hinge well. It is nonetheless used here to designate positions in an Fc region because it is still commonly used in the art to refer to positions in Fc regions. The hinge regions of the IgG1, IgG2, and IgG4 Fc polypeptides extend from about position 216 to about 230. It is clear from the alignment that the IgG2 and IgG4 hinge regions are each three amino acids shorter than the IgG1 hinge. The IgG3 hinge is much longer, extending for an additional 47 amino acids upstream. The CH2 region extends from about position 231 to 340, and the CH3 region extends from about position 341 to 447.

Naturally occurring amino acid sequences of Fc polypeptides can be varied slightly. Such variations can include no more that 10 insertions, deletions, or substitutions of a single amino acid per 100 amino acids of sequence of a naturally occurring Fc polypeptide chain. If there are substitutions, they can be conservative amino acid substitutions, as defined above. The Fc polypeptides on the first and second polypeptide chains can differ in amino acid sequence. In some embodiments, they can include “heterodimerizing alterations,” for example, charge pair substitutions, as defined above, that facilitate heterodimer formation. Further, the Fc polypeptide portions of the heterodimeric antibody can also contain alterations that inhibit FcγR binding. Such mutations are described above and in Xu et at (2000), Cell Immunol. 200(1): 16-26, the relevant portions of which are incorporated herein by reference. The Fc polypeptide portions can also include an “Fc alteration that extends half life,” as described above, including those described in, e.g., U.S. Pat. Nos. 7,037,784, 7,670,600, and 7,371,827, US Patent Application Publication 2010/0234575, and International Application PCT/US2012/070146, the relevant portions of all of which are incorporated herein by reference. Further, an Fc polypeptide can comprise “alterations that enhance ADCC,” as defined above.

A heterodimeric bispecific antibody as described herein can bind to an immune effector cell through an antigen that is part of an effector cell protein and can bind to a target cell through an antigen that is part of a target cell protein. Some effector cell proteins are described in detail below. Similarly, a number of possible target cell proteins is also described below. A heterodimeric bispecific antibody can bind to any combination of an effector cell protein and a target cell protein, which can be engaged noncovalently by the bispecific heterodimeric antibody.

### Nucleic Acids Encoding Heterodimeric Bispecific Antibodies

Provided are nucleic acids encoding the heterodimeric bispecific antibodies described herein. Numerous nucleic acid sequences encoding immunoglobulin regions including VH, VL, hinge, CH1, CH2, CH3, and CH4 regions are known in the art. See, e.g., Kabat et at in SEQUENCES OF IMMUNOLOGICAL INTEREST, Public Health Service N.I.H., Bethesda, MD, 1991. Using the guidance provided herein, one of skill in the art could combine such nucleic acid sequences and/or other nucleic acid sequence known in the art to create nucleic acid sequences encoding the heterodimeric bispecific antibodies described herein. Exemplary pairs of nucleic acids encoding heterodimeric bispecific antibodies include the following: SEQ ID NOs:32 and 33; SEQ ID NOs:34 and 35; SEQ ID NOs:36 and 37; SEQ ID NOs:38 and 39.

In addition, nucleic acid sequences encoding heterodimeric bispecific antibodies described herein can be determined by one of skill in the art based on the amino acid sequences provided herein and knowledge in the art. Besides more traditional methods of producing cloned DNA segments encoding a particular amino acid sequence, companies such as DNA 2.0 (Menlo Park, Calif., USA) and BlueHeron (Bothell, Wash., USA), among others, now routinely produce chemically synthesized, gene-sized DNAs of any desired sequence to order, thus streamlining the process of producing such DNAs.

### Methods of Making the Heterodimeric Bispecific Antibodies

The heterodimeric bispecific antibodies described herein can be made using methods well known in the art. For example, nucleic acids encoding the two polypeptide chains of a heterodimeric bispecific antibody can be introduced into a cultured host cell by a variety of known methods, such as, for example, transformation, transfection, electroporation, bombardment with nucleic acid-coated microprojectiles, etc. In some embodiments the nucleic acids encoding the heterodimeric bispecific antibodies can be inserted into a vector appropriate for expression in the host cells before being introduced into the host cells. Typically such vectors can contain sequence elements enabling expression of the inserted nucleic acids at the RNA and protein levels. Such vectors are well known in the art, and many are commercially available. The host cells containing the nucleic acids can be cultured under conditions so as to enable the cells to express the nucleic acids, and the resulting heterodimeric bispecific antibodies can be collected from the cell mass or the culture medium. Alternatively, the heterodimeric bispecific antibodies can be produced in vivo, for example in plant leaves (see, e.g., Scheller et al. (2001), Nature Biotechnol. 19: 573-577 and references cited therein), bird eggs (see, e.g., Zhu et al. (2005), Nature Biotechnol. 23: 1159-1169 and references cited therein), or mammalian milk (see, e.g., Laible et al. (2012), Reprod. Fertil. Dev. 25(1): 315).

A variety of cultured host cells can be used including, for example, bacterial cells such as Escherichia coli or Bacills steorothermophi/us, fungal cells such as Saccharomyces cerevisiae or Pichia pastoris, insect cells such as lepidopteran insect cells including Spodoptera frugiperda cells, or mammalian cells such as Chinese hamster ovary (CHO) cells, baby hamster kidney (BHK) cells, monkey kidney cells, HeLa cells, human hepatocellular carcinoma cells, or 293 cells, among many others.

### Immune Effector Cells and Effector Cell Proteins

A heterodimeric bispecific antibody as described herein can bind to a molecule expressed on the surface of an immune effector cell (called “effector cell protein” herein) and to another molecule expressed on the surface of a target cell (called a “target cell protein” herein). The immune effector cell can be a T cell, an NK cell, a macrophage, or a neutrophil. In some embodiments the effector cell protein is a protein included in the T cell receptor (TCR)-CD3 complex. The TCR-CD3 complex is a heteromultimer comprising a heterodimer comprising TCRα and TCRβ or TCRγ and TCRδ plus various CD3 chains from among the CD3 zeta (CD3ζ) chain, CD3 epsilon (CD3ε) chain, CD3 gamma (CD3γ) chain, and CD3 delta (CD3δ) chain. In some embodiments, a heterodimeric bispecific antibody binds to a CD3ε chain (the mature amino acid sequence of which is disclosed in SEQ ID NO:40), which may be part of a multimeric protein. Alternatively, the effector cell protein can be human and/or cynomolgus monkey TCRα, TCRβ, TCRδ, TCRγ, CD3 beta (CD3β) chain, CD3γ chain, CCD3δ chain, or CD3ζ chain.

Moreover, in some embodiments, the heterodimeric bispecific antibody can also bind to a CD3ε chain from another species, such as mouse, rat, rabbit, new world monkey, and/or old world monkey species. Such species include, without limitation, the following mammalian species: Mus musculus; Rattus rattus; Rattus norvegicus; the cynomolgus monkey, Macaca fascicularis; the hamadryas baboon, Papio hamadryas; the Guinea baboon, Papio papio; the olive baboon, Papio anubis; the yellow baboon, Papio cynocephalus; the Chacma baboon, Papio ursinus; Callithrix jacchus; Saguinus Oedipus, and Saimiri sciureus. The mature amino acid sequence of the CD3ε chain of cynomolgus monkey is provided in SEQ ID NO:41. As is known in the art of development of protein therapeutics, having a therapeutic that can have comparable activity in humans and species commonly used for preclinical testing, such as mice and monkeys, can simplify and speed drug development. In the long and expensive process of bringing a drug to market, such advantages can be critical.

In more particular embodiments, the heterodimeric bispecific antibody can bind to an epitope within the first 27 amino acids of the CD3ε chain, which may be a human CD3ε chain or a CD3ε chain from different species, particularly one of the mammalian species listed above. The epitope that the antibody binds to can be part of an amino acid sequence selected from the group consisting of SEQ ID NO:40 and SEQ ID NO:41. The epitope can contain the amino acid sequence Gln-Asp-Gly-Asn-Glu (SEQ ID NO:81). The advantages of an antibody that binds such an epitope are explained in detail in U.S. Patent Application Publication 2010/183615, the relevant portions of which are incorporated herein by reference. The epitope to which an antibody binds can be determined by alanine scanning, which is described in, e.g., U.S. Patent Application Publication 2010/183615, the relevant portions of which are incorporated herein by reference.

Where a T cell is the immune effector cell, effector cell proteins to which a heterodimeric bispecific antibody can bind include those that are part of a TCR-CD3 complex including, without limitation, the CD3α chain, the CD3β chain, the CD3γ, the CD3δ chain, the CD3ζ chain, the CD3η chain, TCRα, TCRβ, TCRγ, and TCRδ. Where an NK cell or a cytotoxic T cell is an immune effector cell, NKG2D. CD352, NKp46, or CD16a can be an effector cell protein. Where a CD8⁺ T cell is an immune effector cell, 4-1BB can be an effector cell protein. Alternatively, a heterodimeric bispecific antibody could bind to other effector cell proteins expressed on T cells, NK cells, macrophages, or neutrophils.

### Target Cells and Target Cell Proteins Expressed on Target Cells

As explained above, a heterodimeric bispecific antibody as described herein binds to an effector cell protein and a target cell protein. The target cell protein can, for example, be expressed on the surface of a cancer cell, a cell infected with a pathogen, or a cell that mediates and inflammatory or autoimmune condition. In some embodiments, the target cell protein can be highly expressed on the target cell, although this is not required.

Where the target cell is a cancer cell, a heterodimeric bispecific antibody as described herein can bind to a cancer cell antigen as described above. A cancer cell antigen can be a human protein or a protein from another species. For example, a heterodimeric bispecific antibody may bind to a target cell protein from a mouse, rat, rabbit, new world monkey, and/or old world monkey species, among many others. Such species include, without limitation, the following species: Mus musculus; Rattus rattus; Rattus norvegicus; cynomolgus monkey, Macaca fascicularis; the hamadryas baboon, Papio hamadryas; the Guinea baboon, Papio papio; the olive baboon, Paplo anubis; the yellow baboon, Papio cynocephalus; the Chacma baboon, Papio ursinus, Callithrix jacchus, Saguinus oedipus, and Saimiri sclureus.

In some examples, the target cell protein can be a protein selectively expressed on an infected cell. For example, in the case of an HBV or HCV infection, the target cell protein can be an envelope protein of HBV or HCV that is expressed on the surface of an infected cell. In other embodiments, the target cell protein can be gp120 encoded by human immunodeficiency virus (HIV) on HIV-infected cells.

In other aspects, a target cell can be a cell that mediates an autoimmune or inflammatory disease. For example, human eosinophils in asthma can be target cells, in which case, EGF-like module containing mucin-like hormone receptor (EMR1), for example, can be a target cell protein. Alternatively, excess human B cells in a systemic lupus erythematosus patient can be target cells, in which case CD19 or CD20, for example, can be a target cell protein. In other autoimmune conditions, excess human Th2 T cells can be target cells, in which case CCR4 can, for example, be a target cell protein. Similarly, a target cell can be a fibrotic cell that mediates a disease such as atherosclerosis, chronic obstructive pulmonary disease (COPD), cirrhosis, scleroderma, kidney transplant fibrosis, kidney allograft nephropathy, or a pulmonary fibrosis, including idiopathic pulmonary fibrosis and/or idiotypic pulmonary hypertension. For such fibrotic conditions, fibroblast activation protein alpha (FAP alpha) can, for example, be a target cell protein.

### Target Cell Cytolysis Assays

In the Examples below, an assay for determining whether a heterodimeric bispecific antibody as described herein can induce cytolysis of a target cell by an immune effector cell in vitro is described. In this assay, the immune effector cell is a T cell. The following very similar assay can be used where the immune effector cells are NK cells.

A target cell line expressing the target cell protein of interest can be labeled with 2 μM carboxyfluorescein succnimidyl ester (CFSE) for 15 minutes at 37° C. and then washed. An appropriate number of labeled target cells can then be incubated in one or more 96 well flat bottom culture plates for 40 minutes at 4° C., with or without a bispecific protein, a control protein, or no added protein at varying concentrations. NK cells isolated from healthy human donors can be isolated using the Miltenyi NK Cell Isolation Kit II (Miltenyi Biotec, Auburn, Calif.) and then added to the target cells at an Effector:Target ratio of 10:1. The NK cells, which are the immune effector cells in this assay, can be used immediately post-isolation or after overnight culture at 37° C. Plates containing tumor target cells, bispecific proteins, and immune effector cells can be cultured for 18-24 hours at 37° C. with 5% CO₂. Appropriate control wells can also be set up. After the 18-24 hour assay period, all cells can be removed from the wells. A volume of a 7-AAD solution equal to the volume of the content of the wells can be added to each sample. Samples can then assayed to determine the percentage of live versus dead target cells via flow cytometry as described in the Examples below.

### Therapeutic Methods and Compositions

The heterodimeric bispecific antibodies described herein can be used to treat a wide variety of conditions including, for example, various forms of cancer, infections, fibrotic diseases, and/or autoimmune or inflammatory conditions.

Provided herein are pharmaceutical compositions comprising the heterodimeric bispecific antibodies described herein. Such pharmaceutical compositions comprise a therapeutically effective amount of a heterodimeric bispecific antibody, as described herein, plus one or more additional components such as a physiologically acceptable carrier, excipient, or diluent. Such additional components can include buffers, carbohydrates, polyols, amino acids, chelating agents, stabilizers, and/or preservatives, among many possibilities.

In some embodiments, the heterodimeric, bispecific antibodies described herein can be used to treat cell proliferative diseases, including cancer, which involve the unregulated and/or inappropriate proliferation of cells, sometimes accompanied by destruction of adjacent tissue and growth of new blood vessels, which can allow invasion of cancer cells into new areas, i.e. metastasis. These conditions include hematologic malignancies and solid tumor malignancies. Included within conditions treatable with the heterodimeric bispecific antibodies described herein are non-malignant conditions that involve inappropriate cell growth, including colorectal polyps, cerebral ischemia, gross cystic disease, polycystic kidney disease, benign prostatic hyperplasia, and endometriosis. Other cell proliferative diseases that can be treated using the heterodimeric bispecific antibodies of the present invention are, for example, cancers including mesotheliomas, squamous cell carcinomas, myelomas, osteosarcomas, glioblastomas, gliomas, carcinomas, adenocarcinomas, melanomas, sarcomas, acute and chronic leukemias, lymphomas, and meningiomas, Hodgkin's disease, Sézary syndrome, multiple myeloma, and lung, non-small cell lung, small cell lung, laryngeal, breast, head and neck, bladder, ovarian, skin, prostate, cervical, vaginal, gastric, renal cell, kidney, pancreatic, colorectal, endometrial, and esophageal, hepatobiliary, bone, skin, and hematologic cancers, as well as cancers of the nasal cavity and paranasal sinuses, the nasopharynx, the oral cavity, the oropharynx, the larynx, the hypolarynx, the salivary glands, the mediastinum, the stomach, the small intestine, the colon, the rectum and anal region, the ureter, the urethra, the penis, the testis, the vulva, the endocrine system, the central nervous system, and plasma cells.

Among the texts providing guidance for cancer therapy is Cancer, Principles and Practice of Oncology 4th Edition, DeVita et al, Eds. J. B. Lippincott Co., Philadelphia, Pa. (1993). An appropriate therapeutic approach is chosen according to the particular type of cancer, and other factors such as the general condition of the patient, as is recognized in the pertinent field. The heterodimeric bispecific antibodies described herein may be added to a therapy regimen using other anti-neoplastic agents in treating a cancer patient.

In some embodiments, the heterodimeric bispecific antibodies can be administered concurrently with, before, or after a variety of drugs and treatments widely employed in cancer treatment such as, for example, chemotherapeutic agents, non-chemotherapeutic, anti-neoplastic agents, and/or radiation. For example, chemotherapy and/or radiation can occur before, during, and/or after any of the treatments described herein. Examples of chemotherapeutic agents are discussed above and include, but are not limited to, cisplatin, taxol, etoposide, mitoxantrone (Novantrone®), actinomycin D, cycloheximide, camptothecin (or water soluble derivatives thereof), methotrexate, mitomycin (e.g., mitomycin C), dacarbazine (DTIC), anti-neoplastic antibiotics such as adriamycin (doxorubicin) and daunomycin, and all the chemotherapeutic agents mentioned above.

The heterodimeric bispecific antibodies described herein can also be used to treat infectious disease, for example a chronic hepatis B virus (HBV) infection, a hepatis C virus (HPC) infection, a human immunodeficiency virus (HIV) infection, an Epstein-Barr virus (EBV) infection, or a cytomegalovirus (CMV) infection, among many others.

The heterodimeric bispecific antibodies described herein can find further use in other kinds of conditions where it is beneficial to deplete certain cell types. For example, depletion of human eosinophils in asthma, excess human B cells in systemic lupus erythematosus, excess human Th2 T cells in autoimmune conditions, or pathogen-infected cells in infectious diseases can be beneficial. Depletion of myofibroblasts or other pathological cells in fibrotic conditions such as lung fibrosis, such as idiopathic pulmonary fibrosis (IPF), or kidney or liver fibrosis is a further use of a heterodimeric bispecific antibody.

Therapeutically effective doses of the heterodimeric bispecific antibodies described herein can be administered. The amount of antibody that constitutes a therapeutically dose may vary with the indication treated, the weight of the patient, the calculated skin surface area of the patient. Dosing of the bispecific proteins described herein can be adjusted to achieve the desired effects. In many cases, repeated dosing may be required. For example, a heterodimeric bispecific antibody as described herein can be dosed twice per week, once per week, once every two, three, four, five, six, seven, eight, nine, or ten weeks, or once every two, three, four, five, or six months. The amount of the heterodimeric bispecific antibody administered on each day can be from about 0.0036 mg to about 450 mg. Alternatively, the dose can calibrated according to the estimated skin surface of a patient, and each dose can be from about 0.002 mg/m² to about 250 mg/m². In another alternative, the dose can be calibrated according to a patient's weight, and each dose can be from about 0. 000051 mg/kg to about 6.4 mg/kg.

The heterodimeric bispecific antibodies, or pharmaceutical compositions containing these molecules, can be administered by any feasible method. Protein therapeutics will ordinarily be administered by parenteral route, for example by injection, since oral administration, in the absence of some special formulation or circumstance, would lead to hydrolysis of the protein in the acid environment of the stomach. Subcutaneous, intramuscular, intravenous, intraarterial, intralesional, or peritoneal injection are possible routes of administration. A heterodimeric bispecific antibody can also be administered via infusion, for example intravenous or subcutaneous infusion. Topical administration is also possible, especially for diseases involving the skin. Alternatively, a heterodimeric bispecific antibody can be administered through contact with a mucus membrane, for example by intra-nasal, sublingual, vaginal, or rectal administration or administration as an inhalant. Alternatively, certain appropriate pharmaceutical compositions comprising a heterodimeric bispecific antibody can be administered orally.

Having described the invention in general terms above, the following examples are offered by way of illustration and not limitation.

### EXAMPLES

### Example 1

### Design, Construction, and Production of Heterodimeric Bispecific Antibodies

DNA expression vectors were constructed to produce four different subtypes of heterodimeric bispecific antibodies, which are diagramed in FIG. 1 (2-5), as well as two single chain bispecific molecules, one anti-HER2/CD3ε and one anti-FOLR1/CD3ε. The single chain bispecific molecules contained two VH and two VL regions separated by linkers. Each heterodimeric bispecific antibody contained two polypeptide chains. The first polypeptide chain of each construct comprised two immunoglobulin variable regions followed by a CH1 region and an Fn3 domain that had been engineered to bind albumin, and the second polypeptide chain comprised two immunoglobulin variable regions followed by a CL region. FIG. 1(1).

The coding sequences of immunoglobulin variable regions and constant domains were amplified from DNA templates by polymerase chain reaction (PCR) using forward and reverse primers and subsequently spliced together using a common overhang sequence. See, e.g., Horton et al. (1989), Gene 77: 61-68, the portions of which explain how to perform PCR so as to unite fragments containing matching overhangs is incorporated herein by reference. The PCR products were subcloned into a mammalian expression vector which already contained sequences encoding an albumin-binding fibronection 3 (Fn3) domain (SEQ ID NO:1) and a FLAG®-polyhistidine tag (FLAG-his tag) tag. The Fn3 domain, since it binds to albumin, which is a stable serum protein, is a half-life extending moiety in these constructs. The FLAG-his tag facilitates detection purification.

DNAs encoding the single chain bispecific molecules were made by similar methods. The amino acid sequences of the anti-HER2/CD3 (P136629.3) and anti-FOLR1/CD3 (P136637.3) single chain bispecific molecules are shown in SEQ ID NOs:75 and 76, respectively.

DNA vectors that encode the heterodimeric bispecifc antibodies and single chain bispecific molecules were cotransfected into HEK293-6E cells, and the culture media was harvested after 6 days, concentrated, and buffer-exchanged into IMAC loading buffer. The single chain anti-HER2/CD3 and anti-FOLR1/CD3 molecules were purified by nickel HISTRAP® (GE Healthcare Bio-Sciences, L.L.C., Uppsala, Sweden) column chromatography and eluted with a 25 to 300 mM imidizole gradient. The elution pools were further purified by size exchange chromatography (SEC) using a preparative SUPERDEX® 200 (GE Healthcare Bio-Sciences, L.L.C., Uppsala, Sweden) column, concentrated to >1 mg/mL, and stored at −70° C. The heterodimeric bispecific antibodies were subjected to nickel HISTRAP® (GE Healthcare Bio-Sciences, L.L.C., Uppsala, Sweden) column chromatography and eluted with a 25 to 300 mM imidizole gradient. The elution pools were further purified by size exchange chromatography (SEC) using a preparative SUPERDEX® 200 (GE Healthcare Bio-Sciences, L.L.C., Uppsala, Sweden) column, concentrated to >1 mg/mL, and stored at −70° C.

In an embodiment like that shown in FIG. 1(2) (designated P57216.9), the first polypeptide chain (SEQ ID NO:6) begins with a VH region specific for human MSLN (SEQ ID NO:46), which is followed by a linker, a VH region specific for human CD3c (SEQ ID NO:42), a CH1 region (SEQ ID NO:70), an Fn3 domain engineered to bind to human albumin (SEQ ID NO:1), and a FLAG-his tag. The second polypeptide chain (SEQ ID NO:7) begins with a VL region specific for human MSLN (SEQ ID NO:48), followed by a linker, a VL region specific for human CD3 (SEQ ID NO:43), and a CL region (SEQ ID NO:71). Similarly, SEQ ID NOs: 8 and 9 provide the amino acid sequences of the first and second polypeptide chains, respectively, of another embodiment like that shown in FIG. 1(3) (designated P56019.5). P56019.5 has different variable regions from those used in P57216.9.

An embodiment like that shown in FIG. 1(3) (designated H71362.2) is similar to P56019.5 except that it has different anti-CD3ε variable regions and a different FN3 domain. The anti-CD3ε VH and VL regions in H71362.2 have the amino acid sequences SEQ ID NO:42 and SEQ ID NO:47, respectively, and the first and second polypeptide chains of H71362.2 have the amino acid sequences of SEQ ID NO:10 and SEQ ID NO:11, respectively.

In an embodiment like that shown in FIG. 1(4) (designated P69058.3), the first polypeptide chain (SEQ ID NO:12) begins with a VH region specific for human MSLN (SEQ ID NO:46), which is followed by a linker, a VL region specific for human CD3 (SEQ ID NO:43), a CH1 region, an Fn3 domain (SEQ ID NO:1), and a FLAG-his tag. The second polypeptide chain (SEQ ID NO:13) begins with a VH region specific for human CD3 (SEQ ID NO:42), followed by a linker, a VL region specific for human MSLN (SEQ ID NO:48), and a CL region (SEQ ID NO:73).

In an embodiment like that shown in FIG. 1(5) (designated P69059.3), the first polypeptide chain (SEQ ID NO:14) begins with a VL region specific for human CD3 (SEQ ID NO:43), which is followed by a linker, a VH region specific for human MSLN (SEQ ID NO:46), a CH1 region (SEQ ID NO:70), an Fn3 domain (SEQ ID NO:1), and a FLAG-his tag. The second polypeptide chain (SEQ ID NO:15) begins with a VL region specific for human MSLN (SEQ ID NO:48), followed by a linker, a VH region specific for human CD3 (SEQ ID NO:42), and a CL region (SEQ ID NO:73).

All constructs described above were designed such that interchain interactions between immunoglobulin variable regions were required to create a complete VH/VL antigen-binding pair for each of the two antigens. The linkers between the two immunoglobulin variable regions on each polypeptide chain were short enough, i.e., 5-10 amino acids, that interaction of variable regions on the same polypeptide chains was highly disfavored. In some cases, the first immunoglobulin variable regions on each polypeptide chain could form a complete VH/VL antigen-binding pair, and the second immunoglobulin variable regions on each polypeptide chain could form another VH/VL antigen-binding pair. See FIGS. 1(2) and 1(3) and the description of constructs P56019.5, P57216.9, and H71362.2 above. This kind of interaction is called herein an “in parallel” interaction. In other cases, the first immunoglobulin variable region on the first polypeptide chain could interact with the second immunoglobulin variable region on the second polypeptide chain to form a VH/VL antigen-binding pair, and the second immunoglobulin variable region on the first polypeptide chain could interact with the first immunoglobulin variable region on the second polypeptide chain to form a VH/VL antigen-binding pair. See FIGS. 1(4), 1(5), 1(6) and the descriptions of constructs P69058.3 and P69059.3 above. This kind of interaction is called herein an “diagonal” interaction.

### Example 2

### T Cell Dependent Klling of Cancer Cells by Heterodimeric Bispecifc Antibodies that Bind to MSLN and CD3

The heterodimeric bispecific antibodies described in Example 1 were produced in HEK 293 cells and were assayed by fluorescence activated cell sorting (FACS) for binding to T cells, which express CD3, and to a human ovarian cancer cell line, Ovcar-8, which expresses mesothelin. Briefly, the heterodimeric bispecific antibodies were incubated with about 50,000 Ovcar-8 cells or isolated human or cynomolgus monkey T cells at 4° C. for one hour. The cells were then washed and stained with a fluorescein isothiocyanate (FITC)-conjugated anti-human light chain secondary antibody and analyzed by flow cytometry. The relative binding was represented by the geometric mean of fluorescence intensity. As is apparent in Table 3 below, all constructs tested could bind CD3 on human T cells and MSLN on Ovcar-8 cells.

The anti-MSLN, anti-CD3 heterodimeric bispecific antibodies described in Example 1 were assayed to determine their cytolytic activity against cancer cells expressing MSLN in the presence of human T cells. This assay is referred to herein as the human T cell-dependent cell mediated cytolysis assay (human TDCC). A similar assay using NK cells as immune effector cells is described above. Briefly, a human ovarian cancer line expressing MSLN (Ovcar-8) was labelled with carboxyfluorescein diacetate succinimidyl ester (CFSE) and plated at about 20,000 cells per well in a 96-well V-bottom microtiter plate. Previously frozen isolated human T cells were thawed, washed, and added to the microtiter plate at about 200,000 cells per well. Antibodies were serially diluted to make final well concentrations ranging from 10 μg/mL to 0.01 pg/mL and added to the microtiter plate. Control wells were included which had no antibody, T cells alone, or tumor cells alone. Plates were incubated at 37° C. in a humidified environment for 40 hours. At the end of the assay, all cells from each well were collected (adherent tumor cells were removed using Trypsin-EDTA) and stained using 0.01 pM TO-PRO®-3 (Molecular Probes, Inc., Eugene, Oreg.) to assess viability. Tumor cell viability was read out using flow cytometry. Percent specific lysis was calculated according to the following formula:

% specific lysis=[% tumor cell lysis with bispecific−% tumor cell lysis without bispecific/% of total cell lysis−% tumor cell lysis without bispecific]×100

To determine percent total cell lysis (needed to make this calculation), samples containing effector and labeled target cells without bi-specific were lysed with cold 80% methanol. Results of these assays are summarized in Table 3 below.

TABLE 3
Binding and Cytolytic Activity of Different Subtypes

| | | Amino acid | | | Human TDCC | |
|---|---|---|---|---|---|---|
| | Format as | sequences of the | FACS binding (geometric mean) | | | Maximum |
| Construct | shown in | first and second | Human T | Ovcar-8 | EC₅₀ | killing |
| ID No. | FIG. 1 | polypeptide chains | cells | cells | (pM) | (per cent) |
| P56019.5 | FIG. 1(3) | SEQ ID NO: 8 | 220 | 285 | 0.12 | 53 |
| | | SEQ ID NO: 9 | | | | |
| P57216.9 | FIG. 1(2) | SEQ ID NO: 6 | 103 | 439 | 3.50 | 49 |
| | | SEQ ID NO: 7 | | | | |
| P69058.3 | FIG. 1(4) | SEQ ID NO: 12 | 290 | 588 | <0.1 | 68 |
| | | SEQ ID NO: 13 | | | | |
| P69059.3 | FIG. 1(5) | SEQ ID NO: 14 | 179 | 526 | <0.1 | 68 |
| | | SEQ ID NO: 15 | | | | |
| H71362.2 | FIG. 1(3) | SEQ ID NO: 10 | 354 | 575 | 0.33 | 54 |
| | | SEQ ID NO: 11 | | | | |

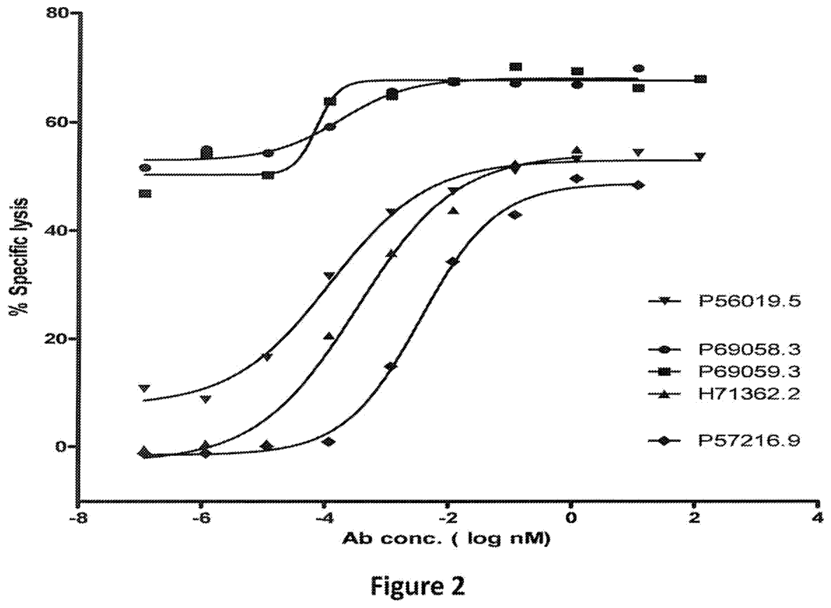

As shown in Table 3, all of the heterodimeric bispecific antibodies tested could bind to human T cells and Ovcar-8 cells. They also exhibited cytolytic activity against tumor cells in the presence of T cells. Table 3 and FIG. 2. However, the two in which diagonal interchain variable regions interactions resulted in complete antigen binding sites, i.e., P69058.3 and P69059.3, had a combination of both low EC₅₀'s and and high maximum killing percents, which was not observed with the other three constructs. These other three constructs were designed such that antigen binding sites could be formed by in parallel interchain interactions between variable regions. These data suggest that constructs requiring an “diagonal” interaction of variable regions may have better biological activity than those requiring in parallel interactions.

Another set of constructs was made by methods similar to those used above using the same pair of anti-MSLN VH and VL regions as used in most constructs described above and a different pair of anti-CD3 VH and VL regions than used in most of the constructs described above. The anti-CD3 VH and VL regions used could bind to both human and cynomolgus monkey CD3. P56019.5 is the only construct described herein using a particular anti-CD3 VH/VL pair that binds to human, but not cynomolgus monkey, CD3. H69070.4 has the same arrangement of variable regions (Le., the format shown in FIG. 1(3)) and the same anti-MSLN VH/VL pair as P56019.5, but it has a different anti-CD3 VH/VL pair, which is also present in H69071.4, H69072.4, and H71365.2. The amino acid sequences of the first and second polypeptide chains of H69070.4 are provided in SEQ ID NO:24 and SEQ ID NO:25 . H69071.4, H69072.4, and H71365.2 all contain the same anti-CD3c VH/VL pair and the same anti-MSLN VH/VL pair, but the variable regions in these constructs are arranged in different ways. See Table 4. The amino acid sequences of first and second polypeptide chains, respectively, of these constructs are as follows: H69071.4, SEQ ID NO:26 and SEQ ID NO:27 ; H69072.4, SEQ ID NO:28 and SEQ ID NO:29 ; and H71364.2, SEQ ID NO:30 and SEQ ID NO:31. These constructs were tested using the assays described above, as well as the cynomolgus monkey T cell-dependent cell cytolysis (called “cyno TDCC”) assay described below.

To perform the cyno TDCC assay, T cells were purified from blood from cynomolgus monkeys as follows. First the red blood cells were lysed with ammonium chloride. Thereafter, the remaining cells were cultured until most of the cultured cells were T cells. These purified cynomolgus monkey T cells were stimulated by incubating them for 48 hrs in a microtiter plate coated with mouse anti-human CD3 in the presence of mouse anti-human CD28. Thereafter, cells were cultured in media containing 10 ng/mL human IL-2 for 7 days. For the assay, a human ovarian cancer line expressing MSLN (Ovcar-8) was CFSE labelled and plated at 10,000 cells per well in a 96-well V-bottom microtiter plate. The stimulated cynomolgus monkey T cells were washed and added to the microtiter plate at 100,000 cells per well. Antibodies were serially diluted 1:10 to make final well concentrations ranging from 10 μg/mL down to 0.01 pg/mL and added to the microtiter plate. Control wells were included that had either no antibody, T cells alone, or tumor cells alone. Microtiter plates were incubated at 37° C. in a humidified environment for 20 hours. At the end of the assay, all cells from each well were collected (adherent tumor cells were removed using Trypsin-EDTA) and stained using 0.01 uM TO-PRO®-3 (Molecular Probes, Inc., Eugene, Oreg.) to assess viability. Tumor cell viability was read out using flow cytometry, and percent specific cell lysis was determined as described above. Results of this assay and those described above are summarized in Table 4 below.

TABLE 4
Binding and Cytolytic Activity of Different Subtypes

| | | Amino acid | | | | Human TDCC | | Cyno TDCC | |
|---|---|---|---|---|---|---|---|---|---|
| | Format as | sequences of the | FACS binding (geometric mean) | | | | Max | | Max |
| Construct | shown in | first and second | Human T | Ovcar-8 | Cyno T | EC₅₀ | killing | EC₅₀ | killing |
| ID No. | FIG. 1 | polypeptide chains | cells | cells | cells | (pM) | (%) | (pM) | (%) |
| P56019.5 | FIG. 1(3) | SEQ ID NO: 8 | 220 | 285 | NA* | 0.12 | 53 | NA | NA |
| | | SEQ ID NO: 9 | | | | | | | |
| H69070.4 | FIG. 1(3) | SEQ ID NO: 24 | 9 | 592 | 127 | 580 | 17 | 3.0 | 88 |
| | | SEQ ID NO: 25 | | | | | | | |
| H69071.4 | FIG. 1(4) | SEQ ID NO: 26 | 16 | 494 | 121 | 6500 | 35 | 3.20 | 88 |
| | | SEQ ID NO: 27 | | | | | | | |
| H69072.4 | FIG. 1(5) | SEQ ID NO: 28 | 11 | 534 | 110 | 44 | 37 | 18.80 | 91 |
| | | SEQ ID NO: 29 | | | | | | | |
| H71365.2 | FIG. 1(3) | SEQ ID NO: 30 | 66 | 558 | 276 | NA* | NA* | 8.10 | 86 |
| | | SEQ ID NO: 31 | | | | | | | |

*“NA” indicates “not applicable,” since activity in the assay was minimal.

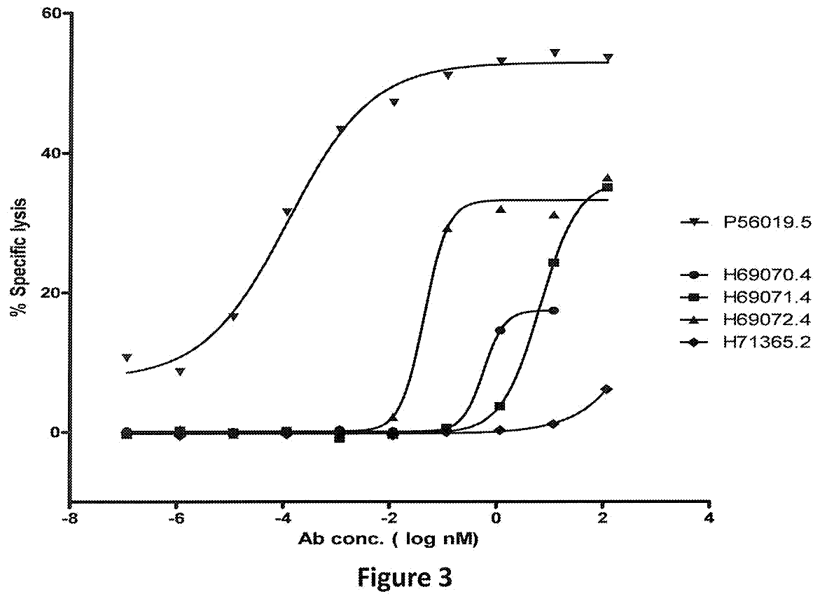

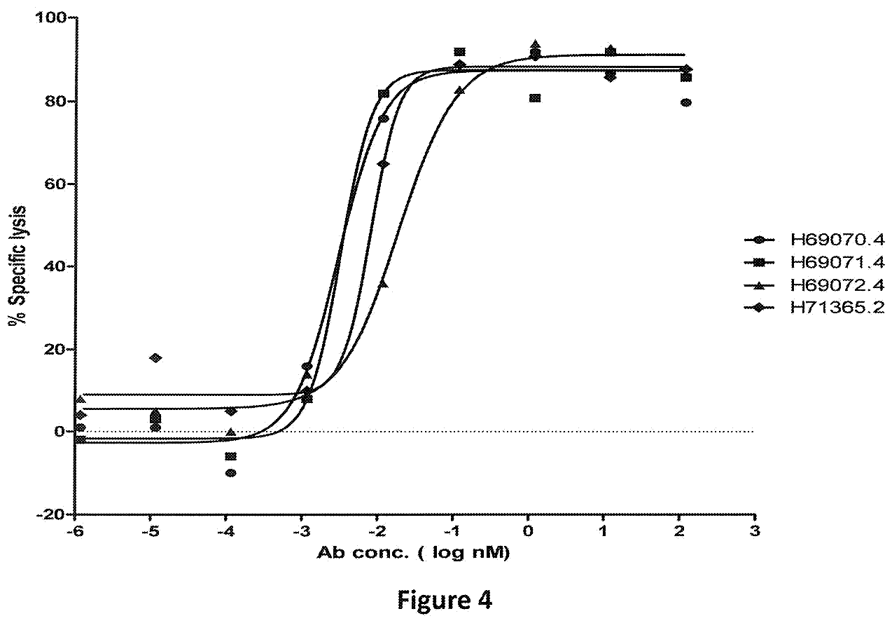

The data in Table 4 indicate that the CD3-binding VH/VL pair used in H69070.4, H69071.2, H69072.4, and H71364.2 binds to cynomolgus monkey CD3, as well as human CD3 to a somewhat lesser extent. Interestingly, construct H69072.4 was much more potent than H69071.4 and H71364.2 (all of which contain the same VH/VL pairs) in the human TDCC assay, although the contructs exhibited roughly comparable activity in the cyno TDCC assay. Table 4 and FIGS. 3 and 4. These data suggest that the particular arrangement of the variable regions in a heterodimeric bispecific antibody can affect its biological activity, perhaps especially in situations where the binding of the variable regions is not particularly robust. For example, the data in Table 4 indicates that most constructs tested did not exhibit as much binding activity for human T cells as they did for cynomolgus monkey T cells. The variable regions were arranged such that interchain interactions resulting in antigen-binding VH/VL pairs were diagonal interactions in constructs H69072.4 and H69071.4. In parallel interactions were required for proper formation of VH/VL pairs in H71365.2. Hence, these data are consistent with the idea that an diagonal interaction of variable regions is more favourable than an in parallel interaction.

### Example 3

### Construction and Characterization of Heterodimeric Bispecific Antibodies Containing an Fc Region

Construct P69058.3 (an anti-MSLN/CD3 heterodimeric bispecific antibody) was modified by the addition of an Fc polypeptide to its second polypeptide chain (containing a CL region) and the replacement of the Fn3 domain in the first polypeptide chain (containing a CH1 region) with an Fc polypeptide. The amino acid sequences of first and second polypeptides of this construct (designated as P73356.3) are provided in SEQ ID NO:16 and SEQ ID NO:17 , respectively. The Fc region in these constructs is a human IgG1 Fc region containing heterodimerizing alterations. Specifically, the first polypeptide chain contains two positively charged mutations (D356K/D399K, using EU numbering as shown in Table 2), and the second polypeptide chain contains two negatively charged mutations (K409D/K392D). These changes result in the preferential formation of heterodimers, as compared to homodimers, when expressed the two polypeptide chains are expressed together in the same cell. See WO 2009/089004. In another construct (P73352.3), the CH1 and CL regions present in P73356.3 in the first and second polypeptide chains, respectively, were removed. The amino acid sequences of the first and second polypeptide chains of P73352.3 are provided in SEQ ID NO:18 and SEQ ID NO:19, respectively.

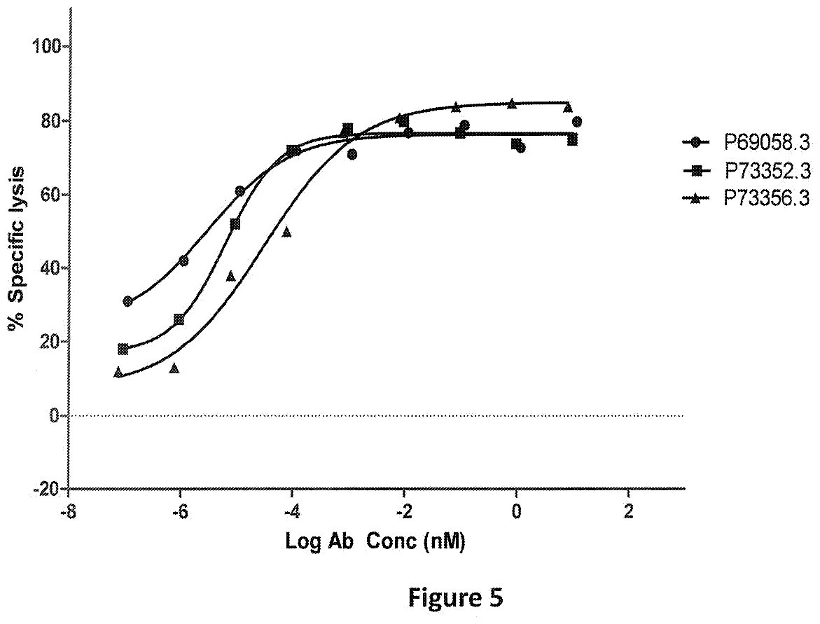

The P73352.3 and P73356.3 constructs were produced in HEK 293 cells and tested together with P69058.3 in a human TDCC assay, as described above. As shown in FIG. 5, both P73352.3 and P73356.3 exhibited potent activity in mediating the killing of Ovcar-8 cells with half-maximum effective concentrations (EC₅₀'s) in subpicomolar range, in the same range as that of P69058.3, which does not contain an Fc region. These data demonstrated the feasibility of generating biologically potent heterodimeric bispecific antibodies that contain an Fc region, with or without the CH and CL regions, and that retain potent T cell-mediated cytolytic activity.

### Example 4

### Heterodimeric Anti-HER2/CD3 Bispecific Antibody Induces Lysis of HER2-Expressing Tumor Cell Lines

Using a format similar to that of 73356.3 (which is in the format of FIG. 1(4) and has an Fc polypeptide chain on the C-terminal end of both the first and second polypeptide chains), P136797.3 was constructed using a VH/VL pair from an anti-HER2 antibody and a VH/VL pair from a different anti-CD3 antibody. The format of P136797.3 is shown in FIG. 1(6). The Fc region of P136797.3 contains additional mutations (L234A/L235A, according to the EU numbering scheme shown in Table 2) to prevent binding to FcγRs. The amino acid sequences of the first and second polypeptide chains of P136797.3 are provided in SEQ ID NO:20 and SEQ ID NO:21 , respectively. An anti-HER2/CD3 single chain bispecific molecule (P136629.3, having the amino acid sequence of SEQ ID NO:75) was also used in the following assay.

Pan T effector cells from human healthy donors were isolated using the Pan T Cell Isolation Kit II, human, Miltenyi Biotec, Auburn, Calif.) and incubated with CFSE-labeled target cells at a ratio of 10:1 (T cell:target cells) in the presence or absence of P136797.3 at varying concentrations. The target cells were either JIMT-1 cells (expressing about 181,000 molecules of HER2 per cell on their cell surface), T47D cells (expressing about 61,000 molecules of HER2 per cell on their cell surface), or SHP77 cells (expressing no detectable HER2 on their cell surface). Following 39-48 hours of incubation, cells were harvested, and tumor cell lysis was monitored by 7AAD uptake using flow cytometry. Percent specific lysis was determined as described in Example 2 above.

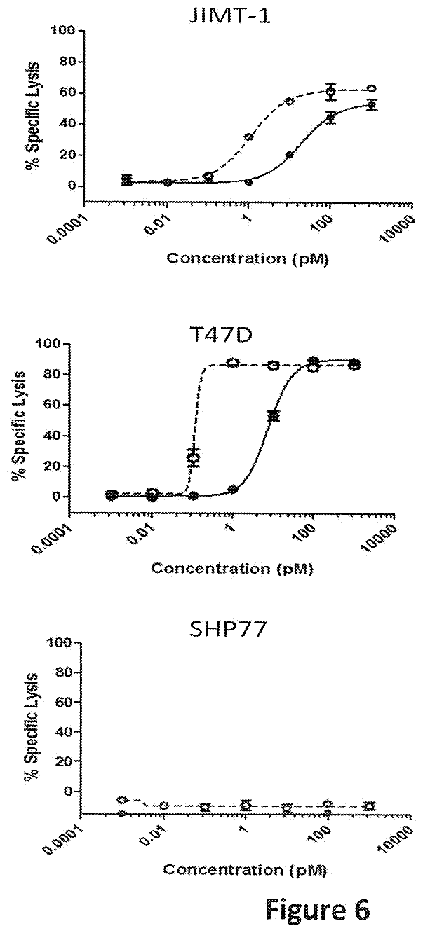

Specific lysis of both JIMT-1 and T47D cells was observed in the presence of appropriate concentrations of P136797.3 or the single chain anti-HER2/CD3 bispecific. The concentration for half maximal lysis (EC₅₀) for P136797.3 was 19.05 pM and 7.75 pM in JIMT-1 and T47D cells, respectively. For the single chain anti-HER2/CD3 bispecific the EC50 was 1.12 pM and 0.12 in JIMT-1 and T47D cells, respectively. There was no specific lysis of the HER2-negative cell line SHP77 observed. FIGS. 6. In addition, lysis of JIMT-1 and T47D cells in the presence of the heterodimeric anti-HER2/CD3 bispecific antibody did not occur in the absence of T cells. Data not shown. These observations suggest that both the heterodimeric anti-HER2/CD3 bispecific antibody and the single chain anti-HER2/CD3 bispecific are a highly specific and potent reagents capable of inducing tumor cell lysis by T cells.

### Example 5

### CD3⁺ Peripheral Blood T Cells in the Presence of PBMC's and a Heterodimeric Bispecific Antibody are Not Activated Unless Target Cells are Present

The following experiment was done to determine whether T cells from peripheral blood could upregulate expression of CD25 and CD69 ex vivo in the presence of the heterodimeric anti-HER2/CD3 bispecific antibody (P136797.3) or the anti-HER2/CD3 single chain bispecific molecule (P136629.3) described above in the presence or absence of HER2-expressing JIMT-1 cells. CD25 and CD69 are considered to be markers for activation of T cells.

Peripheral blood mononuclear cells (PBMC) from healthy donors were purified on a FICOLL™ gradient from human leukocytes purchased from Biological Specialty Corporation of Colmar, Pennsylvania. These PBMC were incubated with P136797.3 or the single chain bispecific molecule at varying concentrations in the absence and presence of the HER2-expressing JIMT-1 tumor cell line. In each sample containing JIMT-1 cells, the ratio of PBMC:JIMT-1 cells was 10:1. Following 48 hours of incubation, non-adherent cells were removed from the wells and divided into two equal samples. Flow cytometry staining was performed to detect the percent of CD3⁺ T cells expressing CD25 or CD69. All samples were stained with a fluorescein isothiocyanate (FITC) conjugated anti-human CD3 antibody. Antibodies against human CD25 and CD69 were allophycocyanin (APC) conjugated. The stained samples were analyzed by FACS.

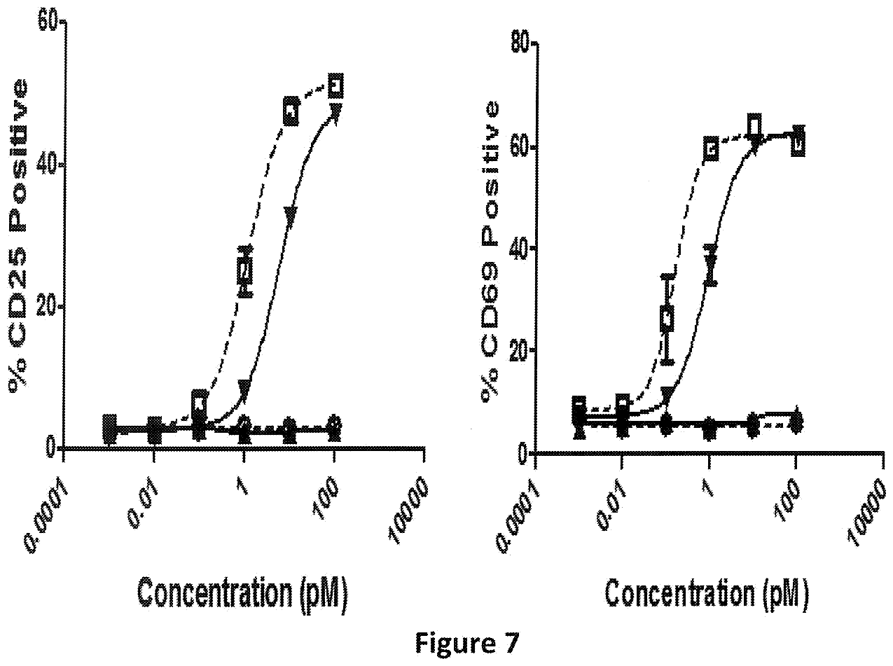

The results are shown in FIG. 7. Up-regulation of CD25 and CD69 in CD3⁺ peripheral T cells was observed with P136797.3 and the single chain bispecific molecule in the presence, but not in the absence, of HER2-expressing JIMT-1 tumor cells. These data indicate that T cell activation by P136797.3 or the single chain bispecific molecule is dependent on the presence of tumor target cells expressing HER2, even though Fc receptor-bearing cells other than T cells are present in PBMC.

### Example 6

### Construction and Testing of an Anti-FOLR1×Anti-CD3 Heterodimeric Bispecific Antibody

In a design similar to that of P136797.3, a heterodimeric bispecific antibody that can bind CD3 and folate receptor 1 (FOLR1), was constructed. It was designated P136795.3. As with P136797.3, the Fc region of P136795.3 contains both charge pair substitutions and mutations blocking binding of FcγR's. The sequences of the first and second polypeptide chains of P136795.3 are provided in SEQ ID NO:22 and SEQ ID NO:23, respectively. An anti-FOLR1/CD3 single chain bispecific molecule (having the amino acid sequence of SEQ ID NO:76) described in Example 1 was also included in this experiment.

Human T cells isolated from healthy donors as described above were incubated with CFSE-labeled tumor target cells at a ratio of 10:1 in the presence and absence of P136795.3. Target cells were either Cal-51 cells (expressing about 148,000 FOLR1 sites/cell), T47D cells (expressing about 101,000 FOLR1 sites/cell), or BT474 cells, which do not express detectable amounts of FOLR1. Following 39-48 hours, cells were harvested and tumor cell lysis was monitored by 7AAD uptake, which stains dead or dying cells but not viable cells, using flow cytometry. Percent specific lysis was determined as described above.

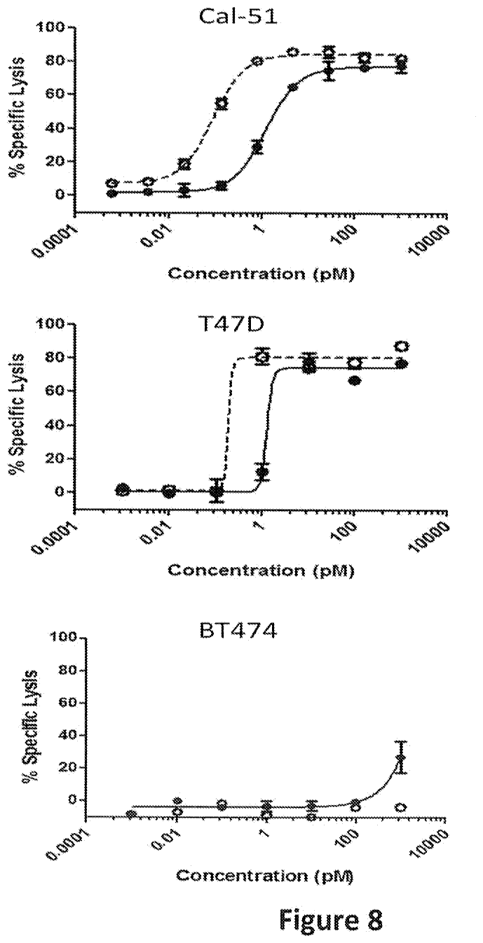

Specific lysis of Cal-51 cells and T47D cells was observed with both P136795.3 and the anti-FOLR1/CD3 single chain bispecific molecule. FIG. 8. The EC₅₀ for P136795.3 was 1.208 pM and 1.26 pM in Cal-61 and T47D cells, respectively. The EC₅₀ for the anti-FOLR1/CD3 single chain bispecific molecule was 0.087 pM and 0.19 pM in Cal-51 and T47D cells, respectively. There was minimal lysis of BT474, a cell line with undetectable levels of FOLR1 (FIGS. 8A), and this lysis was observed only at the highest P136795.3 concentration tested. Tumor target cells in the presence of the P136795.3, but absence of T cells, did not result in 7AAD uptake (data not shown). These observations suggest that both P136795.3 and the anti-FOLR1/CD3 single chain bispecific molecule are a highly specific and potent reagents capable of inducing tumor cell lysis by T cells.

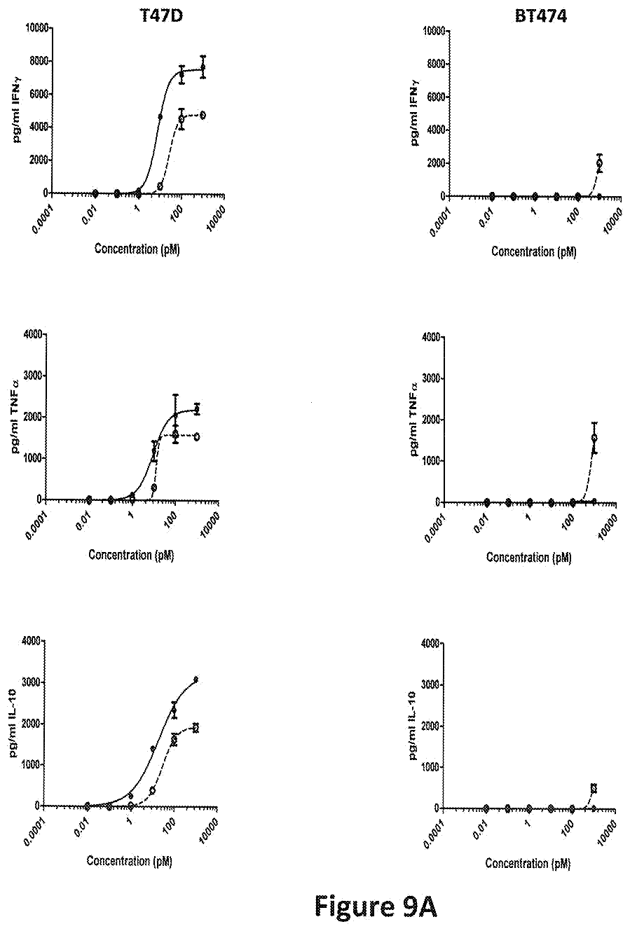

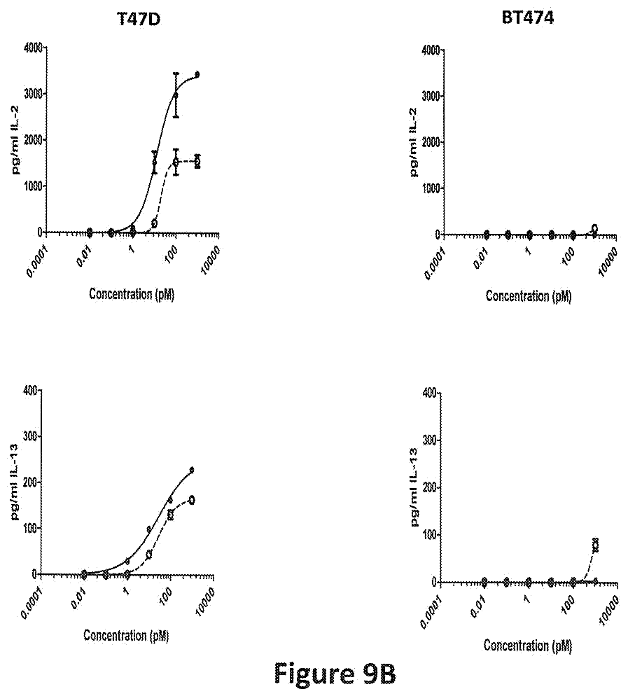

P136795.3 was also tested to determine whether it could stimulate the release of various cytokines by T cells in the presence of a tumor cell line expressing FOLR1 (T47D) or in the presence of a cell line that does not express detectable FOLR1 (BT474). As a positive control, the single chain anti-FOLR1/CD3 bispecific molecule was also tested in this assay. T cells were isolated as described above were incubated in culture medium for about 24 hours in the presence of either T47D cells or BT474 cells in the presence of various concentrations of P136795.3 or the single chain bispecific molecule. The results are shown in FIGS. 9A and 9B. In the presence of T47D cells, the highest cytokine concentrations were seen with IFN-γ, TNF-α, IL-10 and IL-2 (greater than 1000 pg/mL). Moderate levels of IL-13 were also observed. Cytokines were also observed in the presence of the FOLR1-negative cell line, BT474, but only at the highest tested concentration of the heterodimeric bispecific anti-FOLR1/CD3 antibody (1000 pM). The EC₅₀'s for cytokine release in the presence of T47D cells is shown in Table 5 below.

TABLE 5
EC₅₀'s for cytokine release

| | EC₅₀ (pM) for heterodimeric | EC₅₀ (pM) for single chain |
|---|---|---|
| | anti-FOLR1/CD3 | anti-FOLR1/CD3 |
| | in presence of T47D cells | in presence of T47D cells |
| IFN-γ | 27.1 | 7.5 |
| TNF-α | 12.5 | 8.8 |
| IL-10 | 28.3 | 18.4 |
| IL-2 | 20.3 | 12.9 |
| IL-13 | 27.8 | 28.1 |

These results suggest that T cells respond to the presence of an anti-FOLR1/CD3 heterodimeric bispecific antibody or single chain bispecific molecule by secreting cytokines only in the presence of target cells expressing FOLR1.

### Example 7

### HER2-Expressing Cancer Cell-Induced Cytokine Secretion by T Cells

Cell culture supernatants from the TDCC assays as described in Example 4 taken after 24 hours of incubation were assayed for production of various cytokines in the presence of tumor cells expressing HER2 on their cell surface (JIMT-1 cells) or a control cell that did not express the target cell protein (SHP77 cells). Cytokine production by T cells was measured in the presence of an anti-HER2/CD3 heterodimeric bispecific antibody (P136797.3) or single chain bispecific molecule (having the amino acid sequence of SEQ ID NO:75) plus JIMT-1 cells or SHP77 cells. Production of interferon gamma (IFN-γ), tumor necrosis factor alpha (TNF-a), interleukin-10 (IL-10), interleukin-2 (IL-2), and interleukin-13 (IL-13) were measured using the Human TH1/TH2 (7-Plex) Ultra-Sensitive Kit (Catalog No. K15011C-4, Meso Scale Diagnostics, LLC., Rockville, MD) and the Human Proinflammatory I (4-Plex) Ultra-Sensitive Kit (Catalog No. K15009C-4, Meso Scale Diagnostics, LLC., Rockville, Md.) according to the manufacturer's instructions. In the presence of HER2-expressing JIMT-1 cells, T cells treated with P136797.3 or the single chain bispecific molecule released cytokines. Table 6 below shows the EC₅₀ for the five cytokines assayed.

TABLE 6
Cytokine release by T cells in the presence of JIMT-1 cells and
anti-HER2/CD3 bispecific

| | EC₅₀ | |
|---|---|---|
| | JIMT-1 cells | |
| | heterodimeric | single chain anti- |
| cytokine | antiHER2/CD3 | HER2/CD3 |
| IFN-y | 45.5 | 2.1 |
| TNF-a | 36.3 | 1.8 |
| IL-10 | 11.1 | 0.9 |
| IL-2 | 21.5 | 1.2 |
| IL-13 | 19.0 | 1.8 |

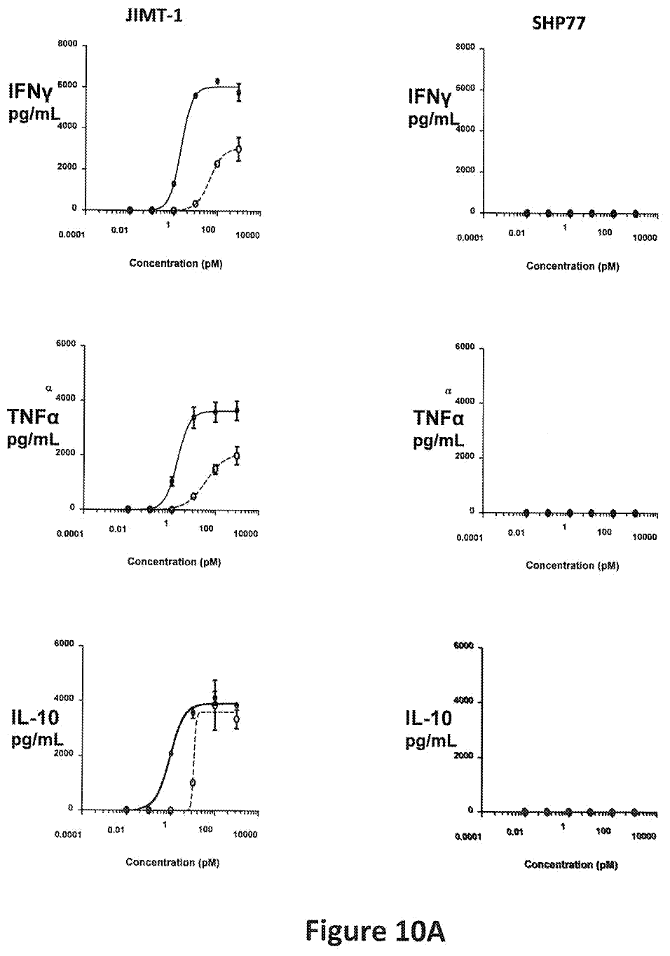

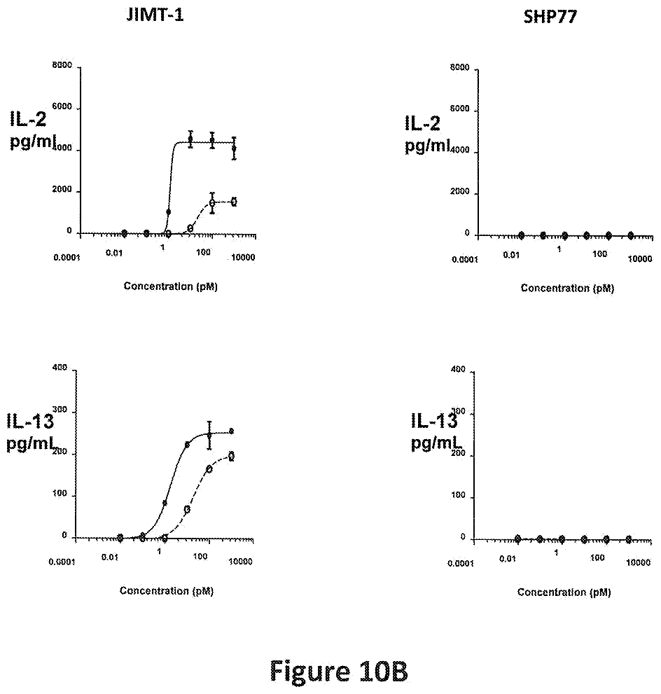

FIGS. 10A and 10B show the titration curves for cytokine production by T cells in the presence of either HER2-expressing JIMT-1 cells or SHP77 cells (which do not express HER2) and varying concentration of P136797.3 or the single chain bispecific molecule. These data indicate that both the anti-HER2/CD3 heterodimeric bispecific antibody and the anti-HER2/CD3 single chain bispecific molecule can induce cytokine production in the presence of JIMT-1 cells, but not in the presence of SHP77 cells.

### Example 8

### In Vivo Activity of a Heterodimeric Bispecific Antibody

The experiment described below demonstrates the activity of a heterodimeric bispecific antibody in an in vivo cancer model system. Humanized mice were generated as follows. One to four days after birth, NOD.Cg-Prkdc^{scid}IL2rg^{tm1}Wjl/SzJ mice (called NSG mice) were irradiated with a dose of 113 centi-Gray (cGY) using a gamma cell irradiator, and about 50,000 previously frozen human CD34⁺ umbilical cord cells were injected into the liver. Starting at 5 weeks of age, animals received 3 weekly intraperitoneal injections of 9 μg of recombinant human IL-7 and 15 μg mouse anti-human IL-7 (a non-neutralizing half-life extending antibody). Blood levels of human T cells were analyzed for each mouse using flow cytometry at 11 weeks of age. Animals used in the study described below had human T cell levels ranging from 0.1% to 40% (relative to all live white blood cells). An additional group of non-humanized, age matched animals (called “control mice”) was included as a control group in the study. These animals (“NSG control mice”) were dosed with P56019.5 (an anti-MSLN/anti-CD3 heterodimeric bispecific antibody) as described below.

For the tumor study, each mouse was implanted subcutaneously with about 10 million cells from a mesothelian-expressing human pancreatic tumor cell line, Capan-2. Treatments were administered intravenously starting nine days after the tumor cell implant. Animals received either (1) five daily injections starting at day 9 of at 100 pg/mouse of P56019.5 (an anti-MSLN/anti-CD3 heterodimeric bispecific antibody), a control bispecific antibody (anti-human EGFRviii/anti-human CD3), or Dulbecco's phosphate buffered saline (DPBS) or (2) two injections, spaced four days apart at 100 pg/mouse, of an anti-human MSLN IgG1 antibody having the same VH and VL regions present in P56019.5 starting at day 9. Tumor volumes were measured, and animals were euthanized when their tumor reached 2000 mm³ or at the end of the study (Day 33). Analysis of the data after completion of the study showed a direct correlation between tumor regression and human T cell numbers, with an apparent minimum of 3% human T cells in the blood being required for activity. Therefore, animals with less than 3% were excluded from the final analysis for all humanized mouse groups resulting in a final animal number of 4 mice per treatment group.

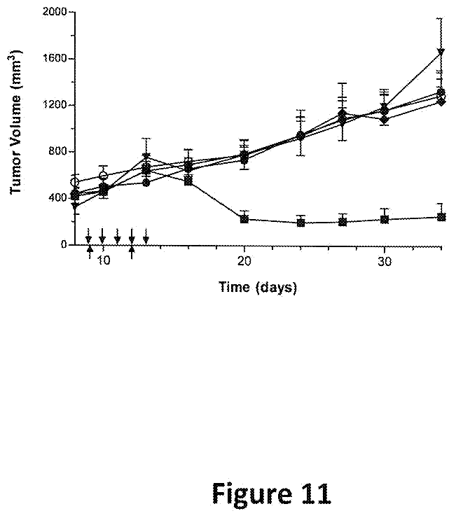

As shown in FIG. 11, implanted Capan-2 cells formed tumors in the “NSG control mice” (which were not humanized) despite treatment with P56019.5. Similarly, tumors formed in mice treated with the anti-human MSLN IgG1 antibody. The control anti-EGFRvIII/CD3 bispecific antibody also could not inhibit the tumor growth. In contrast, tumor growth was significantly suppressed in the humanized mice that were treated with P56019.5 (the anti MSLN/CD3 heterodimeric bispecific antibody). Thus, these data suggest that tumor growth inhibition was dependent on the presence of human T cells and the engagement of both tumor cells and T cells with a bispecific molecule. It further suggests that the T cell dependent suppression of tumor growth is mediated by the binding of mesothelin on Capan-2 cells. This study demonstrated that bispecific heterodimeric antibodies could induce T cell-mediated killing of target cells in vivo.

### Example 9

### Pharmacokinetic Properties of a Heterodimeric Bispecific Antibody

In the experiment described below, the single dose pharmacokinetic properties of a heterodimeric bispecific antibody were compared to those of a single chain bispecific molecule. The first and second polypeptide chains of a heterodimeric bispecific antibody (which was designated P1367973) had the amino acid sequences of SEQ ID NO:20 and SEQ ID NO:21, respectively. The single chain bispecific antibody contained two VH/VL pairs joined by linker, and it had the amino acid sequence of SEQ ID NO:75.

The two test antibodies were injected at a concentration of 1 mg/kg either intravenously via the lateral tail vein in some NOD.SCID mice (obtained from Harlan Laboratories, Livermore, Calif.) or subcutaneously under the skin over the shoulders in others. Approximately 0.1 mL of whole blood was collected at each time point via retro-orbital sinus puncture. Upon clotting of whole blood, the samples were processed to obtain serum (˜0.040 mL per sample). Serum samples were analyzed by immunoassay using the technology Gyros AB (Warren, N.J.) to determine the serum concentrations of the single chain bispecific antibody and heterodimeric bispecific antibody. The assay employed anti-human Fc antibody to capture and detect the heterodimeric bispecific antibody (which contained an Fc region) and a CD3-mimicking peptide to capture the single chain heterodimeric molecule, which was detected with an anti-HIS antibody. Serum samples were collected at 0, 0.5, 2, 8, 24, 72, 120, 168, 240, 312, 384, and 480 hours after injection and maintained at −70° C. (±10° C.) prior to analysis. Pharmacokinetic parameters were estimated from serum concentrations by non-compartmental analysis using Phoenix® 6.3 software (Pharsight, Sunnyvale, Calif.).

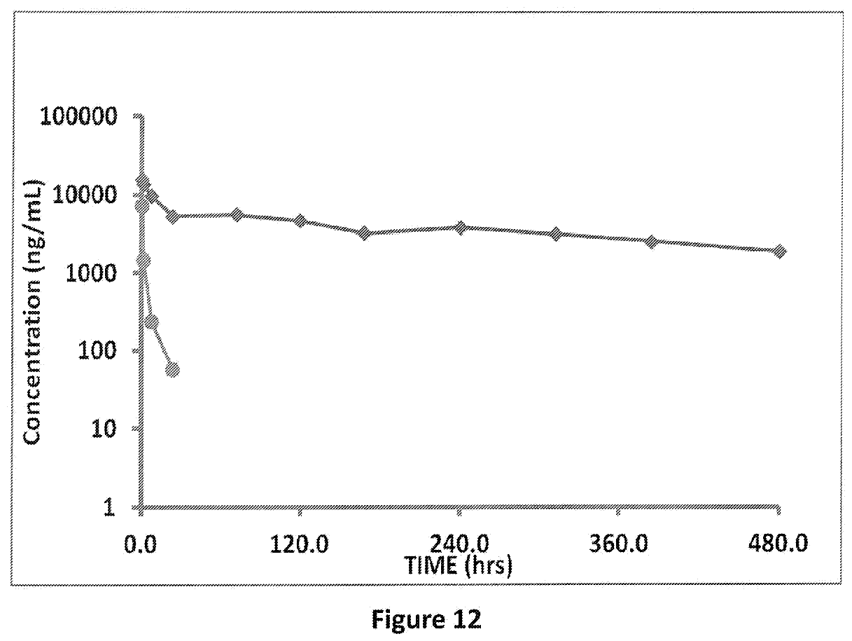

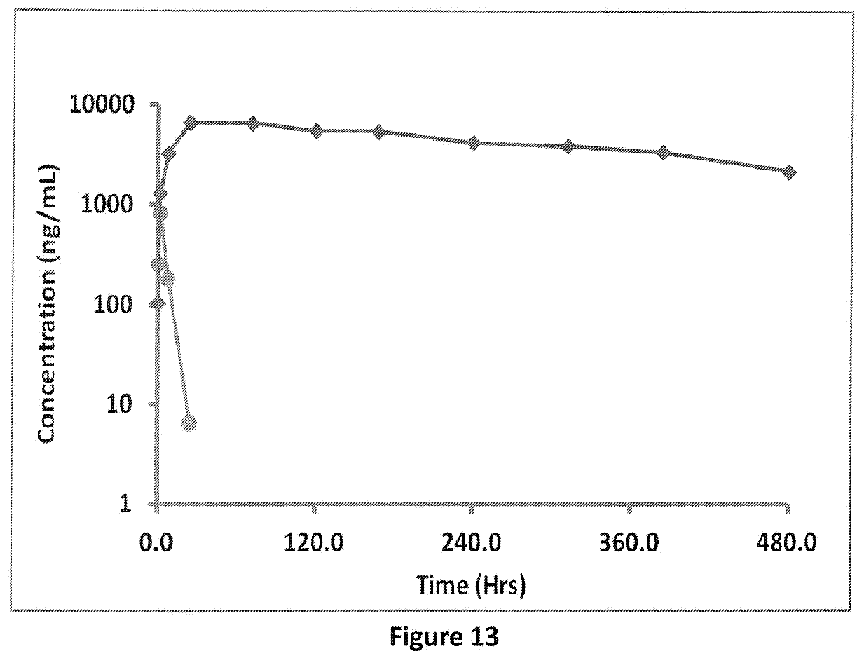

The heterodimeric bispecific antibody showed extended serum half life (223 hours) compared to that of the single chain bispecific antibody (5 hours) when injected either subcutaneously or intravenously. FIGS. 12 and 13. Exposure to the single chain bispecific molecule was characterized by an area under the curve (AUC) of 19 hr*μg/mL, whereas the AUC of the heterodimeric bispecific antibody was 2541 hr*μg/mL. Thus, the heterodimeric bispecific antibody had favorable pharmacokinetic properties

1. A heterodimeric bispecific antibody comprising
(a) a first polypeptide chain comprising an amino acid sequence having the formula V1-L1-V2-L2-CH1, wherein V1 and V2 are immunoglobulin variable regions, L1 and L2 are linkers, L2 can be present or absent, and CH1 is a first immunoglobulin heavy chain constant region; and
(b) a second polypeptide chain comprising an amino acid sequence having the formula V3-L3-V4-L4-CL, wherein V3 and V4 are immunoglobulin variable regions, L3 and L4 are linkers, L4 can be present or absent, and CL is an immunoglobulin light chain constant region;
wherein either or both of the first and the second polypeptide chains further comprise(s) a half life-extending moiety downstream from the regions of (a) and (b); and
wherein the heterodimeric bispecific antibody binds to an immune effector cell and a target cell.

2. The heterodimeric bispecific antibody of claim 1,
wherein the first and second polypeptide chains each comprise an Fc polypeptide chain downstream from the regions recited in (a) and (b), and
wherein the Fc polypeptide chains of the first and second polypeptide chains are human IgG1, IgG2, or IgG4 Fc polypeptide chains.

3. (canceled)

4. The heterodimeric bispecific antibody of claim 1,
wherein the target cell is a cancer cell and the immune effector cell is a T cell, and
wherein the heterodimeric bispecific antibody can mediate increased expression of CD25 and CD69 on the T cell in the presence of target cells, but not in the absence of target cells.

5. The heterodimeric bispecific antibody of claim 2, wherein L1 and L3 are no more than 12 amino acids long.

6. The heterodimeric bispecific antibody of claim 2, wherein one of V1 and V4 is an immunoglobulin heavy chain variable (VH) region and the other is an immunoglobulin light chain variable (VL) region and one of V2 and V3 is a VH region and the other is a VL region, and wherein:
(1) V1 and V4 can bind to a target cell when they are part of an IgG or and/or an scFv antibody and V2 and V3 can bind to an immune effector cell when they are part of an IgG and/or an scFv antibody; or
(2) V1 and V4 can bind to an immune effector cell when they are part of an IgG and/or an scFv antibody and V2 and V3 can bind to a target cell when they are part of an IgG and/or an scFv antibody.

7. The heterodimeric bispecific antibody of claim 6, wherein
(i) V1 and V3 are VL regions and V2 and V4 are VH regions,
(ii) V1 and V3 are VH regions and V2 and V4 are VL regions,
(iii) V1 and V2 are VL regions and V3 and V4 are VH regions, or
(iv) V1 and V2 are VH regions and V3 and V4 are VL regions.

8. The heterodimeric bispecific antibody of claim 2, wherein one of V1 and V3 is a VH region and the other is a VL region and one of V2 and V4 is a VH region and the other is a VL region, and wherein:
(1) V1 and V3 can bind to a target cell when they are part of an IgG and/or an scFv antibody and V2 and V4 can bind to an immune effector cell when they are part of an IgG and/or an scFv antibody, or
(2) V1 and V3 can bind an immune effector cell when they are part of an IgG and/or an scFv antibody and V2 and V4 can bind to a target cell when they are part of an IgG and/or an scFv antibody.

9. The heterodimeric bispecific antibody of claim 8, wherein
(i) V1 and V2 are VH regions and V3 and V4 are VL regions,
(ii) V1 and V2 are VL regions and V3 and V4 are VH regions,
(iii) V1 and V4 are VH regions and V2 and V3 are VL regions, or
(iv) V1 and V4 are VL regions and V2 and V3 are VH regions.

10. The heterodimeric bispecific antibody of claim 2, wherein the effector cell expresses an effector cell protein that is part of a human T cell receptor (TCR)-CD3 complex.

11. The heterodimeric bispecific antibody of claim 10, wherein the effector cell protein is the CD3E chain.

12. The heterodimeric bispecific antibody of claim 11, comprising a VH region comprising the amino acid sequence of SEQ ID NO:42, 44, or 82 or a variant of SEQ ID NO:42, 44, or 82 containing not more than 20 insertions, deletions, or substitutions relative to SEQ ID NO:42, 44, or 82 and a VL region comprising the amino acid sequence of SEQ ID NO:43, 45, or 83 or a variant of SEQ ID NO:43, 45, or 83 containing not more than 20 insertions, deletions, or substitutions of a single amino acid relative to SEQ ID NO:43, 45, or 83.

13. (canceled)

14. The heterodimeric bispecific antibody of claim 2, wherein each Fc-polypeptide chain comprises at least one charge pair substitution.

15. The heterodimeric bispecific antibody of claim 14, wherein:
(1) the Fc polypeptide chain portion of the first polypeptide chain comprises the charge pair substitutions D356K and D399K and the Fc polypeptide chain portion of the second polypeptide comprises the charge pair substitutions K409D and K392D, or
(2) the Fc polypeptide chain portion of the second polypeptide chain comprises the charge pair substitutions D356K and D399K and the Fc polypeptide chain portion of the first polypeptide comprises the charge pair substitutions K409D and K392D.

16. The heterodimeric bispecific antibody of claim 14, wherein the Fc polypeptide chain portions of the first and second polypeptide chains comprise one or more alteration(s) that inhibit(s) Fc gamma receptor (FcγR) binding and/or one or more alteration(s) that extend(s) half life.

17. One or more nucleic acid(s) encoding a heterodimeric bispecific antibody comprising:
(a) a first polypeptide chain comprising an amino acid sequence having the formula V1-L1-V2-L2-CH1, wherein V1 and V2 are immunoglobulin variable regions, L1 and L2 are linkers, L2 can be present or absent, and CH1 is a first immunoglobulin heavy chain constant region; and
(b) a second polypeptide chain comprising an amino acid sequence having the formula V3-L3-V4-L4-CL, wherein V3 and V4 are immunoglobulin variable regions, L3 and L4 are linkers, L4 can be present or absent, and CL is an immunoglobulin light chain constant region;
wherein either or both of the first and the second polypeptide chains further comprise(s) a half life-extending moiety downstream from the regions recited in (a) and (b); and
wherein the heterodimeric bispecific antibody binds to an immune effector cell and a target cell.

18. One or more vector(s) comprising the nucleic acid(s) of claim 17.

19. A host cell comprising the nucleic acid(s) of claim 17.

20. A method of making a heterodimeric bispecific antibody comprising
(1) culturing the host cell of claim 19 under conditions to express the heterodimeric bispecific antibody.

21. A method of treating patient suffering from cancer, an infectious disease, an autoimmune disease, an inflammatory disease, or a fibrotic condition comprising administering to the patient a therapeutically effective amount of a heterodimeric bispecific antibody, wherein the heterodimeric bispecific antibody comprises:
(a) a first polypeptide chain comprising an amino acid sequence having the formula V1-L1-V2-L2-CH1, wherein V1 and V2 are immunoglobulin variable regions, L1 and L2 are linkers, L2 can be present or absent, and CH1 is a first immunoglobulin heavy chain constant region; and
(b) a second polypeptide chain comprising an amino acid sequence having the formula V3-L3-V4-L4-CL, wherein V3 and V4 are immunoglobulin variable regions, L3 and L4 are linkers, L4 can be present or absent, and CL is an immunoglobulin light chain constant region;
wherein both of the first and the second polypeptide chains further comprise an Fc polypeptide chain downstream from the regions of (a) and (b);
wherein the heterodimeric bispecific antibody binds to an immune effector cell and a target cell.

22-24. (canceled)

25. A pharmaceutical composition comprising the heterodimeric bispecific antibody of claim 1.
